SEQUENCE LISTING (4 sequences)

SEQ ID NO: 1 - DNA, 1197 bp, Homo sapiens
  misc_feature (111)..(111): n is a, c, g, or t
atgccgccttctgtctcgtggggcatcctcctgctggcaggcctgtgctgcctggtccctgtctccctggctgaggatccccagggagatgctgcccagaagacagatacntcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggcgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacaggggccatgtttttagaggccatacccatgtctatcccccccgaggtcaagttcaacaaaccctttgtcttcttaatgattgaacaaaataccaagtatcc

SEQ ID NO: 2 - protein, 392 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQATTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDRGHVFRGHTHVYPPRGQVQQTLCLLND

SEQ ID NO: 3 - DNA, 21 bp, Homo sapiens
acaggggccatgtttttagag

SEQ ID NO: 4 - DNA, 21 bp, Homo sapiens
caggggccatgtttttagagg

(19) United States
(12) Patent Application Publication
(10) Pub. No.: US 2010/0048680 A1
(43) Pub. Date: Feb. 25, 2010

(54) Alpha-1-Antitrypsin Variants and Uses Thereof

(75) Inventors: Chen Liu, Gainesville, FL (US); Hui-Jia Dong, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: University of Florida Research Foundation Inc., Gainesville, FL (US)

(21) Appl. No.: 12/522,958

(22) PCT Filed: Jan. 11, 2008
(86) PCT No.: PCT/US08/50895

§ 371 Date: Jul. 24, 2009

Related U.S. Application Data
(60) Provisional application No. 60/871,307, filed on Jan. 11, 2007.

(51) Int. Cl. A61K 31/7088; C12N 5/071; C12Q 1/68; G01N 33/53; A61P 1/16; A61P 37/06

(52) U.S. Cl. 514/44 R; 435/325; 435/6; 435/7.1

(57) ABSTRACT

The subject invention is directed to novel polynucleotides and polypeptides comprising SEQ ID NOs: 1 and 2. Also provided arc fragments these polypeptides. The polynucleotides and polypeptides disclosed herein have been isolated from the liver cells (hepatocytes) of end stage liver failure patients and appear to be associated with a poor prognosis for these patients as relates to liver function. The subject application provides therapeutic methods and reagents for treating livers in which the polynucleotide and polypeptide of SEQ ID NO: 1 and 2 are identified as well as diagnostic methods and reagents for identifying individuals at risk of liver failure. Finally, the subject invention also provides a system of the classification, revision or reordering of a classification system of liver transplant patients.

### CROSS-REFERENCE TO RELATED APPLICATION

This application claims the benefit of U.S. Provisional Application Ser. No. 60/871,307, filed Jan. 11, 2007, the disclosure of which is hereby incorporated by reference in its entirety, including all figures, tables and amino acid or nucleic acid sequences.

Alpha-1-antitrypsin (AAT) is a member of the serpine proteinase inhibitor family. Its main function is to protect tissue from the damage caused by various proteinases during inflammatory responses. The liver is the main source of AAT and deficiency in AAT causes both lung and liver diseases. There is no effective treatment available, except for symptomatic control and replacement therapy.

The prototype of AAT deficiency (PiZZ) affects 1 in 1,800 live births in Northern European and North American populations. The disease is associated with mutation of the gene, AAT. The Z form of AAT is a mutation that results from the substitution of lysine for glutamate at position 342, and accounts for the defective secretion and mutant molecule accumulation in the endoplasmic reticulum of hepatocytes. In ZZ homozygotes, the low serum level of AAT predisposes the patients to lung disease, such as emphysema. In a subgroup of AAT deficiency patients, liver diseases also occur. These liver diseases include chronic hepatitis, cirrhosis, and hepatocellular carcinoma. In fact, AAT deficiency-associated liver disease is the most common genetic liver disease in children and the most common genetic diagnosis for liver transplantation. However, the pathogenesis of the liver disease is poorly understood.

We have identified a truncated form of AAT RNA in liver cells of AAT deficiency patients (designated “DF-AAT”). DF-AAT appears to accumulate in liver cells and appears to be related to the occurrence and severity of liver disease in patients.

### BRIEF SUMMARY OF THE INVENTION

The subject invention is directed to novel polynucleotides and polypeptides comprising SEQ ID NOs: 1 and 2. Also provided are fragments these polypeptides. The polynucleotides and polypeptides disclosed herein have been isolated from the liver cells (hepatocytes) of end stage liver failure patients and appear to be associated with a poor prognosis for these patients as relates to liver function.

The subject application provides therapeutic methods and reagents for treating livers in which the polynucleotide and polypeptide of SEQ ID NO: 1 and 2 are identified as well as diagnostic methods and reagents for identifying individuals at risk of liver failure. Finally, the subject invention also provides a system of the classification, revision or reordering of a classification system of liver transplant patients.

### BRIEF DESCRIPTION OF THE FIGURE

FIG. 1. Polyclonal rabbit anti-DFA antibody was generated and used for the identification of DF-AAT expressed by cells. A Western Blot analysis shows that the antibody specifically recognizes DF-AAT but not wild type/naturally occurring AAT. Lane 1: CHO cells transfected with a plasmid expressing AAT wild type; Lanes 2, 3, and 4: CHO cells transfected with a plasmid expressing DF-AAT, at 48 hrs (lane 2), 72 hrs (lane 3) and 96 hrs (lane 4), respectively. The lane entitled MW provides: the standard molecular weight marker.

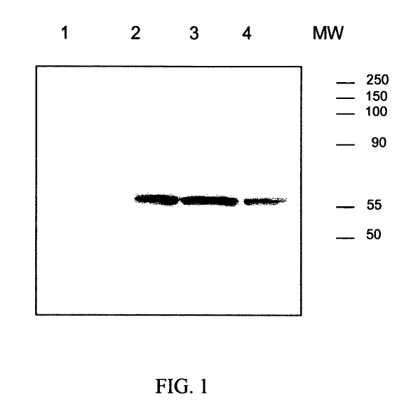

### BRIEF DESCRIPTION OF THE SEQUENCES

SEQ ID NO: 1 is a cDNA encoding the polypeptide of SEQ ID NO: 2.

SEQ ID NO: 2 is a polypeptide that appears to be a splice variant of alpha-1-antitrypsin and is found only in the liver cells (hepatocytes) of end stage liver failure patients.

SEQ ID NOs: 3 and 4 are siRNA sequences derived from the polynucleotide of SEQ ID NO: 1.

### DETAILED DISCLOSURE OF THE INVENTION

The subject application provides the following non-limiting compositions of matter as well as methods of using these compositions of matter. Thus, the subject invention provides various compositions of matter comprising:

a) isolated, purified, and/or recombinant polypeptides comprising SEQ ID NO: 2 or an isolated, purified and/or recombinant polypeptide that is at least 93.15% identical to the polypeptide of SEQ ID NO: 2 (over the full length of SEQ ID NO: 2);

b) a fragment of the polypeptide set forth in SEQ ID NO: 2 or a fragment of SEQ ID NO: 2 that is “from Y to Z”, wherein Y is the N-terminal amino acid of the specified sequence and Z is the C-terminal amino acid of the specified sequence with the proviso that at least one of the amino acids found at positions 366 through 392 is contained within said fragment. Thus, for SEQ ID NO: 2, each fragment can be between 5 consecutive amino acids and 391 consecutive amino acids in length and each fragment containing between 5 and 391 consecutive amino acids of SEQ ID NO: 2 is specifically contemplated by the subject invention. Fragments “from Y to Z”, wherein Y is the N-terminal amino acid of the specified sequence and Z is the C-terminal amino acid of a specified sequence are provided in Table 1 for SEQ ID NO: 2. Polypeptide fragments as set forth in this application have at least one biological activity that is substantially the same as the corresponding biological activity of the full-length polypeptide of SEQ ID NO: 2;

c) a polypeptide according to any one of embodiments a) or b) that further comprises a heterologous polypeptide sequence;

d) a composition comprising a carrier and a polypeptide as set forth in any one of a), b) or c), wherein said carrier is an adjuvant or a pharmaceutically acceptable excipient;

e) a polynucleotide sequence: i) encoding a polypeptide comprising SEQ ID NO: 2; ii) encoding one or more polypeptide fragment of SEQ ID NO: 2 as set forth in (b); or iii) encoding a polypeptide as set forth in (b) or (c);

f) a polynucleotide sequence that is at least 91.50% identical to SEQ ID NO: 1 (over the full length of SEQ ID NO: 1);

g) a polynucleotide sequence comprising SEQ ID NO: 1, 3 or 4;

h) a polynucleotide sequence that is at least 8 consecutive nucleotides of a polynucleotide sequence as set forth in (e), (f) or (g) or a polynucleotide as set forth in Table 3 or Table 4;

i) a polynucleotide that is fully complementary to the polynucleotides set forth in (e), (f), (g) or (h);

j) a polynucleotide that hybridizes under low, intermediate or high stringency with a polynucleotide sequence as set forth in (e), (f), (g), (h) or (i);

k) a genetic construct comprising a polynucleotide sequence as set forth in (e), (f), (g), (h), (i), or (j);

l) a vector comprising a polynucleotide or genetic construct as set forth in (e), (f), (g), (h), (i), (j), (k) or (l);

m) a host cell comprising a vector as set forth in (l), a genetic construct as set forth in (k), or a polynucleotide as set forth in any one of (e), (f), (g), (h), (i) or (j);

n) a probe comprising a polynucleotide according to (g), (h), (i), (j), (k) or (l) and, optionally, a label or marker;

o) an antisense nucleic acid comprising a sequence fully complementary to the polynucleotide of SEQ ID NO: 1, a fragment of SEQ ID NO: 1 that includes or spans a least one nucleotide at positions 1095 to 1197 of SEQ ID NO: 1 and is at least 8 nucleotides in length, or a span of nucleotides as set forth in Table 3 or Table 4;

p) a siRNA molecule comprising SEQ ID NO: 3 or 4.

In the context of the instant invention, the terms “oligopeptide”, “polypeptide”, “peptide” and “protein” can be used interchangeably; however, it should be understood that the invention does not relate to the polypeptides in natural form, that is to say that they are not in their natural environment but that the polypeptides may have been isolated or obtained by purification from natural sources or obtained from host cells prepared by genetic manipulation (e.g., the polypeptides, or fragments thereof, are recombinantly produced by host cells, or by chemical synthesis). Polypeptides according to the instant invention may also contain non-natural amino acids, as will be described below. The terms “oligopeptide”, “polypeptide”, “peptide” and “protein” are also used, in the instant specification, to designate a series of residues, typically L-amino acids, connected one to the other, typically by peptide bonds between the α-amino and carboxyl groups of adjacent amino acids. Linker elements can be joined to the polypeptides of the subject invention through peptide bonds or via chemical bonds (e.g., heterobifunctional chemical linker elements) as set forth below. Additionally, the terms “amino acid(s)” and “residue(s)” can be used interchangeably.

In the context of both polypeptides and polynucleotides, the term “successive” can be used interchangeably with the term “consecutive” or the phrase “contiguous span” throughout the subject application. Thus, in some embodiments, a polynucleotide fragment may be referred to as “a contiguous span of at least X nucleotides, wherein X is any integer value beginning with 5; the upper limit for fragments as set forth herein is one nucleotide less than the total number of nucleotides found in the full-length sequence encoding a particular polypeptide (e.g., a polypeptide comprising SEQ ID NO: 2). A polypeptide fragment, by example, may be referred to as “a contiguous span of at least X amino acids, wherein X is any integer value beginning with 5; the upper limit for such polypeptide fragments is one amino acid less than the total number of amino acids found in the full-length sequence of a particular polypeptide (e.g., 392 for SEQ ID NO: 2). As used herein, the term “integer” refers to whole numbers in the mathematical sense.

“Nucleotide sequence”, “polynucleotide” or “nucleic acid” can be used interchangeably and are understood to mean, according to the present invention, either a double-stranded DNA, a single-stranded DNA or products of transcription of the said DNAs (e.g., RNA molecules). It should also be understood that the present invention does not relate to genomic polynucleotide sequences in their natural environment or natural state. The nucleic acid, polynucleotide, or nucleotide sequences of the invention can be isolated, purified (or partially purified), by separation methods including, but not limited to, ion-exchange chromatography, molecular size exclusion chromatography, or by genetic engineering methods such as amplification, subtractive hybridization, cloning, subcloning or chemical synthesis, or combinations of these genetic engineering methods.

The terms “comprising”, “consisting of” and “consisting essentially of” are defined according to their standard meaning. The terms may be substituted for one another throughout the instant application in order to attach the specific meaning associated with each term. The phrases “isolated” or “biologically pure” refer to material that is substantially or essentially free from components which normally accompany the material as it is found in its native state. Thus, isolated peptides in accordance with the invention preferably do not contain materials normally associated with the peptides in their in situ environment. “Link” or “join” refers to any method known in the art for functionally connecting peptides, including, without limitation, recombinant fusion, covalent bonding, disulfide bonding, ionic bonding, hydrogen bonding, and electrostatic bonding.

Thus, the subject invention provides polypeptides comprising SEQ ID NO: 2 and/or polypeptide fragments of SEQ ID NO: 2. Polypeptide fragments, according to the subject invention, comprise a contiguous span of at least 5 consecutive amino acids of SEQ ID NO: 2 and the include at least one amino acid found at positions 366 through 392 of SEQ ID NO: 2. Polypeptide fragments according to the subject invention can be any integer in length from at least 5 consecutive amino acids to 1 amino acid less than a full length polypeptide of SEQ ID NO: 2. Thus, fragments of SEQ ID NO: 2 can contain any number (integer) of consecutive amino acids between, and including, 5 and 391.

Each polypeptide fragment of the subject invention can also be described in terms of its N-terminal and C-terminal positions. Additionally, polypeptide fragments embodiments described herein may be “at least”, “equal to”, “equal to or less than”, “less than”, “at least ______ but not greater than ______” or “from Y to Z”, wherein Y is the N-terminal amino acid of the specified sequence and Z is the C-terminal amino acid of the specified sequence, the fragment is at least 5 amino acids in length, and Y and Z are any integer specified (or selected from) those integers identified in the tables specifying the corresponding fragment lengths for each polypeptide disclosed herein (see Table 1 [the positions listed in the tables correspond to the amino acid position as provided in the attached sequence listing]). As is apparent from Table 1, the N-terminal amino acid for fragments of SEQ ID NO: 2 can be any integer from 1 to 388 and the C-terminal amino acid is any integer from 5 to 391 (depending on the fragment length which is to be is any number (integer) of consecutive amino acids between, and including, 5 and 391).

The subject invention also provides for various polypeptide fragments (comprising contiguous spans or consecutive spans of at least five consecutive amino acids) that span particular residues of SEQ ID NO: 2. In the context of this invention, the polypeptide fragments span at least one of the amino acids found at positions 366 through 392 of SEQ ID NO: 2.

Fragments, as described herein, can be obtained by cleaving the polypeptides of the invention with a proteolytic enzyme (such as trypsin, chymotrypsin, or collagenase) or with a chemical reagent, such as cyanogen bromide (CNBr). Alternatively, polypeptide fragments can be generated in a highly acidic environment, for example at pH 2.5. Such polypeptide fragments may be equally well prepared by chemical synthesis or using hosts transformed with an expression vector according to the invention. The transformed host cells contain a nucleic acid, allowing the expression of these fragments, under the control of appropriate elements for regulation and/or expression of the polypeptide fragments.

In certain preferred embodiments, fragments of the polypeptides disclosed herein retain at least one biological property or biological activity of the full-length polypeptide from which the fragments are derived (such fragments may also be referred to as “biologically active fragments”. Thus, both full length polypeptides and fragments of the polypeptides provided by SEQ ID NO: 2 have one or more of the following properties or biological activities: the ability to: 1) specifically bind to antibodies specific for SEQ ID NO: 2, wherein said antibodies do not bind to known alpha-1-antitrypsin precursor proteins; or 2) the polypeptides or fragments are associated with liver cells (hepatocytes) that are in end stage failure.

The polypeptides (or fragments thereof) of the invention may be monomeric or multimeric (e.g., dimers, trimers, tetramers and higher multimers). Accordingly, the present invention relates to monomers and multimers of the polypeptides of the invention, their preparation, and compositions containing them. Multimeric polypeptides, as set forth herein, may be formed by hydrophobic, hydrophilic, ionic and/or covalent associations and/or may be indirectly linked, by for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention arc formed by covalent associations with and/or between the polypeptides of the invention. One non-limiting example of such a covalent association is the formation disulfide bonds between immunoglobulin heavy chains as provided by a fusion protein of the invention that comprises a polypeptide comprising SEQ ID NO: 2 (or fragments thereof) fused to an Ig heavy chain (see, e.g., U.S. Pat. No. 5,478,925, which disclosure is hereby incorporated by reference in its entirety). Another example of a fusion protein capable of forming covalently associated multimers is oseteoprotegerin (see, e.g., International Publication No. WO 98/49305, the contents of which is incorporated by reference in its entirety). In another embodiment, two or more polypeptides of the invention are joined through peptide linkers. Examples include those peptide linkers described in U.S. Pat. No. 5,073,627 (hereby incorporated by reference). Proteins comprising multiple polypeptides of the invention separated by peptide linkers may be produced using conventional recombinant DNA technology.

Other multimeric polypeptides can be formed by fusing the polypeptides of the invention to a leucine zipper or isoleucine zipper polypeptide sequence. Leucine zipper and isoleucine zipper domains are polypeptides that promote multimerization of the proteins in which they are found. Non-limiting examples of leucine zipper domains suitable for producing soluble multimeric proteins of the invention are those described in PCT application WO 94/10308, hereby incorporated by reference. Recombinant fusion proteins comprising a polypeptide of the invention fused to a polypeptide sequence that dimerizes or trimerizes in solution are expressed in suitable host cells, and the resulting soluble multimeric fusion protein is recovered from the culture supernatant using techniques known in the art.

Multimeric polypeptides can also be generated using chemical techniques known in the art. For example, polypeptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimeric polypeptides can be generated by introducing disulfide bonds between the cysteine residues located within the sequence of the polypeptides that are being used to construct the multimeric polypeptide (see, e.g., U.S. Pat. No. 5,478,925, which is herein incorporated by reference in its entirety). Further, polypeptides of the invention may be routinely modified by the addition of cysteine or biotin to the C terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, other techniques known in the art may be applied to generate liposomes containing the polypeptide components desired to be contained in the multimer of the invention (see, e.g., U.S. Pat. No. 5,478,925, which is herein incorporated by reference in its entirety).

The polypeptides provided herein, as well as the fragments thereof, may further comprise linker elements (L) that facilitate the attachment of the fragments to other molecules, amino acids, or polypeptide sequences. The linkers can also be used to attach the polypeptides, or fragments thereof, to solid support matrices for use in affinity purification protocols. Non-limiting examples of “linkers” suitable for the practice of the invention include chemical linkers (such as those sold by Pierce, Rockford, Ill.), or peptides that allow for the connection combinations of polypeptides (see, for example, linkers such as those disclosed in U.S. Pat. Nos. 6,121,424, 5,843,464, 5,750,352, and 5,990,275, hereby incorporated by reference in their entirety).

In other embodiments, the linker element (L) can be an amino acid sequence (a peptide linker). In some embodiments, the peptide linker has one or more of the following characteristics: a) it allows for the free rotation of the polypeptides that it links (relative to each other); b) it is resistant or susceptible to digestion (cleavage) by proteases; and c) it does not interact with the polypeptides it joins together. In various embodiments, a multimeric construct according to the subject invention includes a peptide linker and the peptide linker is 5 to 60 amino acids in length. More preferably, the peptide linker is 10 to 30, amino acids in length; even more preferably, the peptide linker is 10 to 20 amino acids in length. In some embodiments, the peptide linker is 17 amino acids in length.

Peptide linkers suitable for use in the subject invention are made up of amino acids selected from the group consisting of Gly, Ser, Asn, Thr and Ala. Preferably, the peptide linker includes a Gly-Ser element. In a preferred embodiment, the peptide linker comprises (Ser-Gly-Gly-Gly-Gly)y wherein y is 1, 2, 3, 4, 5, 6, 7, or 8. Other embodiments provide for a peptide linker comprising ((Ser-Gly-Gly-Gly-Gly)_{y}-Ser-Pro). In certain preferred embodiments, y is a value of 3, 4, or 5. In other preferred embodiment, the peptide linker comprises (Ser-Ser-Ser-Ser-Gly)_{y} or ((Ser-Ser-Ser-Ser-Gly)_{y}-Ser-Pro), wherein y is 1, 2, 3, 4, 5, 6, 7, or 8. In certain preferred embodiments, y is a value of 3, 4, or 5. Where cleavable linker elements are desired, one or more cleavable linker sequences such as Factor Xa or enterokinase (Invitrogen, San Diego Calif.) can be used alone or in combination with the aforementioned linkers.

Multimeric constructs of the subject invention can also comprise a series of repeating elements, optionally interspersed with other elements. As would be appreciated by one skilled in the art, the order in which the repeating elements occur in the multimeric polypeptide is not critical and any arrangement of the repeating elements as set forth herein can be provided by the subject invention. Thus, a “multimeric construct” according to the subject invention can provide a multimeric polypeptide comprising a series of polypeptides or polypeptide fragments that are, optionally, joined together by linker elements (either chemical linker elements or amino acid linker elements).

Fusion proteins according to the subject invention comprise one or more heterologous polypeptide sequences (e.g., tags that facilitate purification of the polypeptides of the invention (see, for example, U.S. Pat. No. 6,342,362, hereby incorporated by reference in its entirety; Altendorf et al., (1999-WWW, 2000); Baneyx, (1999); Eihauer et al., (2001); Jones et al., (1995); Margolin (2000); Puig et al., (2001); Sassenfeld (1990); Sheibani (1999); Skerra et al., (1999); Smith (1998); Smyth et al., (2000); Unger (1997), each of which is hereby incorporated by reference in their entireties), or commercially available tags from vendors such as such as STRATAGENE (La Jolla, Calif.), NOVAGEN (Madison, Wis.), QIAGEN, Inc., (Valencia, Calif.), or InVitrogen (San Diego, Calif.).

In other embodiments, polypeptides of the subject invention (e.g., SEQ ID NO: 2 or fragments thereof) can be fused to heterologous polypeptide sequences that have adjuvant activity (a polypeptide adjuvant). Non-limiting examples of such polypeptides include heat shock proteins (hsp) (see, for example, U.S. Pat. No. 6,524,825, the disclosure of which is hereby incorporated by reference in its entirety).

The subject application also provides a composition comprising at least one isolated, recombinant, or purified polypeptide comprising SEQ ID NO: 2 (or a fragment thereof) and at least one additional component. In various aspects of the invention, the additional component is a solid support (for example, microtiter wells, magnetic beads, non-magnetic beads, agarose beads, glass, cellulose, plastics, polyethylene, polypropylene, polyester, nitrocellulose, nylon, or polysulfone). The additional component can also be a pharmaceutically acceptable excipient or adjuvant known to those skilled in the art. In some aspects of the invention, the solid support provides an array of polypeptides of the subject invention or an array of polypeptides comprising combinations of various polypeptides of the subject invention.

The subject invention also provides methods for eliciting an immune response in an individual comprising the administration of compositions comprising polypeptides according to the subject invention to an individual in amounts sufficient to induce an immune response in the individual. In some embodiments, the polypeptide of SEQ ID NO: 2 (or fragments thereof) results in the induction of antibody production, or induces a CTL (or CD8⁺ T cell) and/or an HTL (or CD4⁺ T cell), and/or an antibody response that can prevents, reduces or at least partially arrests disease symptoms, side effects or progression of disease in the individuals.

Individuals, in the context of this application, refers to mammals such as, but not limited to, apes, chimpanzees, orangutans, humans, monkeys or domesticated animals (pets) such as dogs, cats, guinea pigs, hamsters, rabbits, ferrets, cows, horses, goats and sheep.

Administering or administer is defined as the introduction of a substance into the body of an individual and includes oral, nasal, ocular, rectal, vaginal and parenteral routes. Compositions may be administered individually or in combination with other agents via any route of administration, including but not limited to subcutaneous (SQ), intramuscular (IM), intravenous (IV), intraperitoneal (IP), intradermal (ID), via the nasal, ocular or oral mucosa (IN), or orally.

The composition administered to the individual may, optionally, contain an adjuvant and may be delivered in any manner known in the art for the delivery of immunogen to a subject. Compositions may also be formulated in any carriers, including for example, pharmaceutically acceptable carriers such as those described in E. W. Martin's Remington's Pharmaceutical Science, Mack Publishing Company, Easton, Pa. In preferred embodiments, compositions may be formulated in incomplete Freund's adjuvant, complete Freund's adjuvant, or alum. Other non-limiting examples of adjuvants that can be used in the practice of the invention include: oil-water emulsions, Polygen, Carbigen (Carbopol 934P) or Titer-Max (Block copolymer CRL-8941, squalene and a unique microparticulate stabilizer).

In other embodiments, the subject invention provides for diagnostic assays based upon Western blot formats or standard immunoassays known to the skilled artisan and which utilize a polypeptide comprising, consisting essentially of, or consisting of SEQ ID NO: 2 or fragments thereof. For example, antibody-based assays such as enzyme linked immunosorbent assays (ELISAs), radioimmunoassays (RIAs), lateral flow assays, reversible flow chromatographic binding assay (see, for example, U.S. Pat. No. 5,726,010, which is hereby incorporated by reference in its entirety), immunochromatographic strip assays, automated flow assays, and assays utilizing peptide-containing biosensors may be employed for the detection of antibodies that bind to the polypeptides (or fragments thereof) that are provided by the subject invention. The assays and methods for conducting the assays are well-known in the art and the methods may test biological samples (e.g., serum, plasma, or blood) qualitatively (presence or absence of antibody (e.g., an autoantibody that specifically binds the polypeptide of SEQ ID NO: 2)) or quantitatively (comparison of a sample against a standard curve prepared using a polypeptide of the subject invention) for the presence of antibodies that bind to polypeptides of the subject invention.

The antibody-based assays can be considered to be of four types: direct binding assays, sandwich assays, competition assays, and displacement assays. In a direct binding assay, either the antibody or antigen is labeled, and there is a means of measuring the number of complexes formed. In a sandwich assay, the formation of a complex of at least three components (e.g., antibody-antigen-antibody) is measured. In a competition assay, labeled antigen and unlabelled antigen compete for binding to the antibody, and either the bound or the free component is measured. In a displacement assay, the labeled antigen is pre-bound to the antibody, and a change in signal is measured as the unlabelled antigen displaces the bound, labeled antigen from the receptor.

Lateral flow assays can be conducted according to the teachings of U.S. Pat. No. 5,712,170 and the references cited therein. U.S. Pat. No. 5,712,170 and the references cited therein are hereby incorporated by reference in their entireties. Displacement assays and flow immunosensors useful for carrying out displacement assays are described in: Kusterbeck et al., (1990); Kusterbeck et al., (1990a); Ligler et al., (1992); Ogert et al., (1992), all of which are incorporated herein by reference in their entireties. Displacement assays and flow immunosensors are also described in U.S. Pat. No. 5,183,740, which is also incorporated herein by reference in its entirety. The displacement immunoassay, unlike most of the competitive immunoassays used to detect small molecules, can generate a positive signal with increasing antigen concentration.

The subject invention also provides methods of binding an antibody to a polypeptide of the subject invention (e.g., SEQ ID NO: 2, or an antibody binding fragment thereof) comprising contacting a sample containing an antibody with a polypeptide under conditions that allow for the formation of an antibody-antigen complex. These methods can further comprise the step of detecting the formation of said antibody-antigen complex. In various aspects of this method, an immunoassay is conducted for the detecting the presence of the polypeptide in hepatocytes or samples derived from hepatocytes, and predicting the outcome or prognosis of liver disease in an individual. Such an assay can also be used for monitoring the progression of liver disease in an individual, the development of antibodies within the patient being indicative of the onset of end stage liver failure/disease. Non-limiting examples of such immunoassays include enzyme linked immunosorbent assays (ELISAs), radioimmunoassays (RIAs), lateral flow assays, immunochromatographic strip assays, automated flow assays, Western blots, immunoprecipitation assays, reversible flow chromatographic binding assays, agglutination assays, and biosensors. Additional aspects of the invention provide for the use of an array of polypeptides or antibodies specific to the polypeptide of SEQ ID NO: 2 (the array can contain the polypeptide of SEQ ID NO: 2 (or fragments thereof) and/or antibodies that specifically bind to SEQ ID NO: 2).

The subject invention also concerns antibodies that bind to polypeptides of the invention. Antibodies that are immunospecific (specifically bind) the polypeptide of SEQ ID NO: 2 are specifically contemplated. Antibodies of the subject invention do not cross-react with, immunoreact or specifically bind to, other known alpha-1-antitrypsin polypeptides. The antibodies of the subject invention can be prepared using standard materials and methods known in the art (see, for example, Monoclonal Antibodies: Principles and Practice, 1983; Monoclonal Hybridoma Antibodies: Techniques and Applications, 1982; Selected Methods in Cellular Immunology, 1980; Immunological Methods, Vol. II, 1981; Practical Immunology, and Kohler et al., 1975). These antibodies can further comprise one or more additional components, such as a solid support, a carrier or pharmaceutically acceptable excipient, or a label.

The term “antibody” includes monoclonal antibodies (including full length monoclonal antibodies), polyclonal antibodies, multispecific antibodies (e.g., bispecific antibodies), and antibody fragments so long as they exhibit the desired biological activity, particularly the ability to specifically bind to the polypeptide of SEQ ID NO: 2 without cross reacting with other known alpha-1-antitrypsing polypeptides. “Antibody fragments” comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multi-specific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975), or may be made by recombinant DNA methods (see, e.g., U.S. Pat. No. 4,816,567). The “monoclonal antibodies” may also be isolated from phage antibody libraries using the techniques described in Clackson et al. (1991) and Marks et al. (1991), for example.

The monoclonal antibodies described herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., (1984)). Also included are humanized antibodies that specifically bind to the polypeptides, or fragments thereof, set forth in SEQ ID NO: 2 (see, for example, U.S. Pat. Nos. 6,407,213 or 6,417,337, which are hereby incorporated by reference in their entirety, teaching methods of making humanized antibodies).

“Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}−V_{L}). Diabodies are described more fully in, for example, EP 404,097; WO 93/11161; and Holliger et al. (1993). The term “linear antibodies” refers to the antibodies described in Zapata et al. (1995).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As discussed above. “nucleotide sequence”, “polynucleotide” or “nucleic acid” can be used interchangeably and are understood to mean, according to the present invention, either a double-stranded DNA, a single-stranded DNA or products of transcription of said DNAs (e.g., RNA molecules).

Both protein and nucleic acid sequence homologies may be evaluated using any of the variety of sequence comparison algorithms and programs known in the art. Such algorithms and programs include, but are by no means limited to, TBLASTN, BLASTP, FASTA, TFASTA, and CLUSTALW (Pearson et al., 1988; Altschul et al., 1990; Thompson et al., 1994; Higgins et al., 1996; Gish et al., 1993). Sequence comparisons are, typically, conducted using default parameters provided by the vendor or using those parameters set forth in the above-identified references, which are hereby incorporated by reference in their entireties.

The subject invention contemplates polypeptides and polynucleotides having between 90.00% and 99.99% identity to the full length sequences set forth in SEQ ID NO: 1 and 2. The range of identity, between 90.00% and 99.99%, is to be taken as including, and providing written description and support for, any fractional percentage, in intervals of 0.01%, between 90.00% and, up to, including 99.99%. These percentages are purely statistical and differences between two nucleic acid sequences can be distributed randomly and over the entire sequence length. For example, homologous sequences can exhibit a percent identity of 90, 91, 92, 93, 94, 95, 96, 97, 98, or 99 percent with the sequences of the instant invention. As set forth above, the percent identity is, typically, calculated with reference to the full length, native, and/or naturally occurring polynucleotide or polypeptide. The terms “identical” or percent “identity”, in the context of two or more polynucleotide or polypeptide sequences, refer to two or more sequences or subsequences that are the same or have a specified percentage of nucleotides or amino acid residues that are the same, when compared and aligned for maximum correspondence over a comparison window, as measured using a sequence comparison algorithm or by manual alignment and visual inspection.

A “complementary” polynucleotide sequence, as used herein, generally refers to a sequence arising from the hydrogen bonding between a particular purine and a particular pyrimidine in double-stranded nucleic acid molecules (DNA-DNA, DNA-RNA, or RNA-RNA). The major specific pairings are guanine with cytosine and adenine with thymine or uracil. A “complementary” polynucleotide sequence may also be referred to as an “antisense” polynucleotide sequence or an “antisense sequence”. The term “fully complementary” refers to a polynucleotide sequence that hybridizes, without a mismatch, over the full length of a particular nucleic acid sequence.

Sequence homology and sequence identity can also be determined by hybridization studies under high stringency, intermediate stringency, and/or low stringency. Various degrees of stringency of hybridization can be employed. The more severe the conditions, the greater the complementarity that is required for duplex formation. Severity of conditions can be controlled by temperature, probe concentration, probe length, ionic strength, time, and the like. Preferably, hybridization is conducted under low, intermediate, or high stringency conditions by techniques well known in the art, as described, for example, in Keller, G. H., M. M. Manak (1987).

For example, hybridization of immobilized DNA on Southern blots with ³²P-labeled gene-specific probes can be performed by standard methods (Maniatis et al., 1982). In general, hybridization and subsequent washes can be carried out under intermediate to high stringency conditions that allow for detection of target sequences with homology to the exemplified polynucleotide sequence. For double-stranded DNA gene probes, hybridization can be carried out overnight at 20-25° C. below the melting temperature (Tₘ) of the DNA hybrid in 6×SSPE, 5× Denhardt's solution, 0.1% SDS, 0.1 mg/ml denatured DNA. The melting temperature is described by the following formula (Beltz et al., 1983).

Tm=81.5° C.+16.6 Log[Na⁺]+0.41(% G−C)−0.61 (%formamide)-600/length of duplex in base pairs.

Washes are typically carried out as follows:

(1) twice at room temperature for 15 minutes in 1×SSPE, 0.1% SDS (low stringency wash);

(2) once at Tₘ−20° C. for 15 minutes in 0.2×SSPE, 0.1% SDS (intermediate stringency wash).

For oligonucleotide probes, hybridization can be carried out overnight at 10-20° C. below the melting temperature (Tₘ) of the hybrid in 6×SSPE, 5× Denhardt's solution, 0.1% SDS, 0.1 mg/ml denatured DNA. Tₘ for oligonucleotide probes can be determined by the following formula:

Tₘ(° C.)=2(number T/A base pairs)⁺4(number G/C base pairs) (Suggs et al., 1981).

Washes can be carried out as follows:

(1) twice at room temperature for 15 minutes 1×SSPE, 0.1% SDS (low stringency wash);

2) once at the hybridization temperature for 15 minutes in 1×SSPE, 0.1% SDS (intermediate stringency wash).

In general, salt and/or temperature can be altered to change stringency. With a labeled DNA fragment >70 or so bases in length, the following conditions can be used:

Low: 1 or 2×SSPE, room temperature

Low: 1 or 2×SSPE, 42° C.

Intermediate: 0.2× or 1×SSPE, 65° C.

High: 0.1×SSPE, 65° C.

By way of another non-limiting example, procedures using conditions of high stringency can also be performed as follows: Pre-hybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in pre-hybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1×SSC corresponding to 0.15M NaCl and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. Other conditions of high stringency which may be used are well known in the art and as cited in Sambrook et al. (1989) and Ausubel et al. (1989) are incorporated herein in their entirety.

Another non-limiting example of procedures using conditions of intermediate stringency are as follows: Filters containing DNA are pre-hybridized, and then hybridized at a temperature of 60° C. in the presence of a 5×SSC buffer and labeled probe. Subsequently, filters washes are performed in a solution containing 2×SSC at 50° C. and the hybridized probes are detectable by autoradiography. Other conditions of intermediate stringency which may be used are well known in the art and as cited in Sambrook et al. (1989) and Ausubel et al. (1989) are incorporated herein in their entirety.

Duplex formation and stability depend on substantial complementarity between the two strands of a hybrid and, as noted above, a certain degree of mismatch can be tolerated. Therefore, the probe sequences of the subject invention include mutations (both single and multiple), deletions, insertions of the described sequences, and combinations thereof, wherein said mutations, insertions and deletions permit formation of stable hybrids with the target polynucleotide of interest. Mutations, insertions and deletions can be produced in a given polynucleotide sequence in many ways, and these methods are known to an ordinarily skilled artisan. Other methods may become known in the future.

It is also well known in the art that restriction enzymes can be used to obtain functional fragments of the subject DNA sequences. For example, Bal31 exonuclease can be conveniently used for time-controlled limited digestion of DNA (commonly referred to as “erase-a-base” procedures). See, for example, Maniatis et al. (1982).

The present invention further comprises fragments of the polynucleotide sequences of the instant invention. Representative fragments of the polynucleotide sequences according to the invention will be understood to mean any nucleotide fragment having at least 5 successive nucleotides, preferably at least 12 successive nucleotides, and still more preferably at least 15, 18, or at least 20 successive nucleotides of the sequence from which it is derived. The upper limit for fragments as set forth herein is the total number of nucleotides found in the full-length sequence encoding a particular polypeptide (e.g., a polypeptide such as that of SEQ ID NO: 2). Certain non-limiting examples of polynucleotide fragments of the subject invention are provided in Tables 3 and 4. In these tables, the starting position of the fragment (the 5′ end of the polynucleotide fragment as denoted by position “Y”) corresponds to the nucleotide position as described in SEQ ID NO: 1 and the last nucleotide within the fragment (position “Z” as determined according to the formula provided within the table) corresponds to that same position within SEQ ID NO: 1.

In some embodiments, the subject invention includes those fragments capable of hybridizing under various conditions of stringency conditions (e.g., high or intermediate or low stringency) with a nucleotide sequence according to the invention; fragments that hybridize with a nucleotide sequence of the subject invention can be, optionally, labeled as set forth below.

The subject invention provides, in one embodiment, methods for the identification of the presence of nucleic acids according to the subject invention in transformed host cells or in hepatic cells isolated from an individual suspected of being at risk for liver failure. In these varied embodiments, the invention provides for the detection of nucleic acids in a sample (obtained from the individual or from a cell culture) comprising contacting a sample with a nucleic acid (polynucleotide) of the subject invention (such as an RNA, mRNA, DNA, cDNA, or other nucleic acid). In a preferred embodiment, the polynucleotide is a probe that is, optionally, labeled and used in the detection system. Many methods for detection of nucleic acids exist and any suitable method for detection is encompassed by the instant invention. Typical assay formats utilizing nucleic acid hybridization includes, and are not limited to, 1) nuclear run-on assay, 2) slot blot assay, 3) northern blot assay (Alwine et al., 1977), 4) magnetic particle separation, 5) nucleic acid or DNA chips, 6) reverse Northern blot assay, 7) dot blot assay, 8) in situ hybridization, 9) RNase protection assay (Melton et al., 1984) and as described in the 1998 catalog of Ambion, Inc., Austin, Tex.), 10) ligase chain reaction, 11) polymerase chain reaction (PCR), 12) reverse transcriptase (RT)-PCR (Berchtold, 1989), 13) differential display RT-PCR (DDRT-PCR) or other suitable combinations of techniques and assays. Labels suitable for use in these detection methodologies include, and are not limited to 1) radioactive labels, 2) enzyme labels, 3) chemiluminescent labels, 4) fluorescent labels, 5) magnetic labels, or other suitable labels, including those set forth below. These methodologies and labels are well known in the art and widely available to the skilled artisan. Likewise, methods of incorporating labels into the nucleic acids are also well known to the skilled artisan.

Thus, the subject invention also provides primers and detection probes (e.g., fragments of the disclosed polynucleotide sequence) for hybridization with a target sequence or the amplicon generated from the target sequence. Such a primer or detection probe will comprise a contiguous/consecutive span of at least 8, 9, 10, 11, 12, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 nucleotides and will, preferably, include or span at least one nucleotide found at positions 1095 through 1197 of SEQ ID NO: 1. Labeled probes or primers are labeled with a radioactive compound or with another type of label as set forth above (e.g., 1) radioactive labels, 2) enzyme labels, 3) chemiluminescent labels, 4) fluorescent labels, or 5) magnetic labels). Alternatively, non-labeled nucleotide sequences may be used directly as probes or primers; however, the sequences are generally labeled with a radioactive element (³²P, ³⁵S, ³H, ¹²⁵I) or with a molecule such as biotin, acetylaminofluorene, digoxigenin, 5-bromo-deoxyuridine, or fluorescein to provide probes that can be used in numerous applications.

Polynucleotides of the subject invention can also be used for the qualitative and quantitative analysis of gene expression using arrays or polynucleotides that are attached to a solid support. As used herein, the term array means a one -, two-, or multi-dimensional arrangement of full length polynucleotides or polynucleotides of sufficient length to permit specific detection of gene expression. Preferably, the fragments are at least 15, 100, 150, 200, 250, 300, 350, 500, 450 or 500 nucleotides in length and include or span at least one nucleotide found at positions 1095 through 1197 of SEQ ID NO: 1.

For example, quantitative analysis of gene expression may be performed with full-length polynucleotides of the subject invention, or fragments thereof that include or span at least one nucleotide found at positions 1095 through 1197 of SEQ ID NO: 1, in a complementary DNA microarray as described by Schena et al. (1995, 1996). Polynucleotides, or fragments thereof that include or span at least one nucleotide found at positions 1095 through 1197 of SEQ ID NO: 1, are amplified by PCR and arrayed onto silylated microscope slides. Printed arrays are incubated in a humid chamber to allow rehydration of the array elements and rinsed, once in 0.2% SDS for 1 min, twice in water for 1 min and once for 5 min in sodium borohydride solution. The arrays are submerged in water for 2 min at 95° C., transferred into 0.2% SDS for 1 min, rinsed twice with water, air dried and stored in the dark at 25° C.

mRNA is isolated from a biological sample and probes are prepared by a single round of reverse transcription. Probes are hybridized to 1 cm² microarrays under a 14×14 mm glass coverslip for 6-12 hours at 60° C. Arrays are washed for 5 min at 25° C. in low stringency wash buffer (1×SSC/0.2% SDS), then for 10 min at room temperature in high stringency wash buffer (0.1×SSC/0.2% SDS). Arrays are scanned in 0.1×SSC using a fluorescence laser scanning device fitted with a custom filter set. Accurate differential expression measurements are obtained by taking the average of the ratios of two independent hybridizations.

Quantitative analysis of the polynucleotides present in a biological sample can also be performed in complementary DNA arrays as described by Pietu et al. (1996). The polynucleotides of the invention, or fragments thereof, are PCR amplified and spotted on membranes. Then, mRNAs originating from biological samples derived from various tissues or cells are labeled with radioactive nucleotides. After hybridization and washing in controlled conditions, the hybridized mRNAs are detected by phospho-imaging or autoradiography. Duplicate experiments are performed and a quantitative analysis of differentially expressed mRNAs is then performed.

Alternatively, the polynucleotide sequences of to the invention may also be used in analytical systems, such as DNA chips. DNA chips and their uses are well known in the art and (see for example, U.S. Pat. Nos. 5,561,071; 5,753,439; 6,214,545; Schena 1996; Bianchi et al., 1997; each of which is hereby incorporated by reference in their entireties) and/or are provided by commercial vendors such as Affymetrix, Inc. (Santa Clara, Calif.). In addition, the nucleic acid sequences of the subject invention can be used as molecular weight markers in nucleic acid analysis procedures.

The subject invention also provides genetic constructs comprising: a) a polynucleotide sequence encoding a polypeptide comprising SEQ ID NO: 2, or a fragment thereof including or spanning at least one amino acid found at positions 366 through 392 of SEQ ID NO: 2; b) a polynucleotide sequence having at least about 93.15% to 99.99% identity to a polynucleotide sequence encoding a polypeptide comprising SEQ ID NO: 2, or a fragment of SEQ ID NO: 2 including or spanning at least one amino acid found at positions 366 through 392 of SEQ ID NO: 2; c) a polynucleotide sequence encoding a polypeptide having at least about 93.15% to 99.99% identity to a polypeptide comprising SEQ ID NO: 2, or a fragment of SEQ ID NO: 2, optionally including or spanning at least one amino acid found at positions 366 through 392 of SEQ ID NO: 2, or a fragment thereof, d) a polynucleotide sequence comprising SEQ ID NO: 1; e) a polynucleotide sequence having at least about 91.5% to 99.99% identity to the polynucleotide sequence of SEQ ID NO: 1 over the full length of SEQ ID NO: 1; f) a polynucleotide sequence encoding multimeric construct; or g) a polynucleotide that is complementary to the polynucleotides set forth in (a), (b), (c), (d), (e) or (f). Genetic constructs of the subject invention can also contain additional regulatory elements such as promoters and enhancers and, optionally, selectable markers.

Also within the scope of the subject instant invention are vectors or expression cassettes containing genetic constructs as set forth herein or polynucleotides encoding the polypeptides, set forth supra, operably linked to regulatory elements. The vectors and expression cassettes may contain additional transcriptional control sequences as well. The vectors and expression cassettes may further comprise selectable markers. The expression cassette may contain at least one additional gene, operably linked to control elements, to be co-transformed into the organism. Alternatively, the additional gene(s) and control element(s) can be provided on multiple expression cassettes. Such expression cassettes are provided with a plurality of restriction sites for insertion of the sequences of the invention to be under the transcriptional regulation of the regulatory regions. The expression cassette(s) may additionally contain selectable marker genes operably linked to control elements.

The expression cassette will include in the 5′-3′ direction of transcription, a transcriptional and translational initiation region, a DNA sequence of the invention, and a transcriptional and translational termination regions. The transcriptional initiation region, the promoter, may be native or analogous, or foreign or heterologous, to the host cell. Additionally, the promoter may be the natural sequence or alternatively a synthetic sequence. As used herein, a chimeric gene comprises a coding sequence operably linked to a transcriptional initiation region that is heterologous to the coding sequence.

Another aspect of the invention provides vectors for the cloning and/or the expression of a polynucleotide sequence taught herein. Vectors of this invention, including vaccine vectors, can also comprise elements necessary to allow the expression and/or the secretion of the said nucleotide sequences in a given host cell. The vector can contain a promoter, signals for initiation and for termination of translation, as well as appropriate regions for regulation of transcription. In certain embodiments, the vectors can be stably maintained in the host cell and can, optionally, contain signal sequences directing the secretion of translated protein. These different elements are chosen according to the host cell used. Vectors can integrate into the host genome or, optionally, be autonomously-replicating vectors.

The subject invention also provides for the expression of a polypeptide or peptide fragment encoded by a polynucleotide sequence disclosed herein comprising the culture of a host cell transformed with a polynucleotide of the subject invention under conditions that allow for the expression of the polypeptide and, optionally, recovering the expressed polypeptide.

The disclosed polynucleotide sequences can also be regulated by a second nucleic acid sequence so that the protein or peptide is expressed in a host transformed with the recombinant DNA molecule. For example, expression of a protein or peptide may be controlled by any promoter/enhancer element known in the art. Promoters which may be used to control expression include, but are not limited to, the CMV-IE promoter, the SV40 early promoter region (Benoist and Chambon 1981), the promoter contained in the 3′ long terminal repeat of Rous sarcoma virus (Yamamoto et al., 1980), the herpes simplex thymidine kinase promoter, the regulatory sequences of the metallothionein gene; prokaryotic vectors containing promoters such as the β-lactamase promoter (Villa-Kamaroff et al., 1978), or the tac promoter (deBoer et al., 1983); see also “Useful proteins from recombinant bacteria” in Scientific American, 1980, 242:74-94; plant expression vectors comprising the nopaline synthetase promoter region or the cauliflower mosaic virus 35S RNA promoter, and the promoter of the photosynthetic enzyme ribulose biphosphate carboxylase; promoter elements from yeast or fungi such as the Gal 4 promoter, the ADC (alcohol dehydrogenase) promoter, PGK (phosphoglycerol kinase) promoter, and/or the alkaline phosphatase promoter.

The vectors according to the invention are, for example, vectors of plasmid or viral origin. In a specific embodiment, a vector is used that comprises a promoter operably linked to a protein or peptide-encoding nucleic acid sequence contained within the disclosed polynucleotide sequences, one or more origins of replication, and, optionally, one or more selectable markers (e.g., an antibiotic resistance gene). Expression vectors comprise regulatory sequences that control gene expression, including gene expression in a desired host cell. Exemplary vectors for the expression of the polypeptides of the invention include the pET-type plasmid vectors (Promega) or pBAD plasmid vectors (Invitrogen) or those provided in the examples below. Furthermore, the vectors according to the invention are useful for transforming host cells so as to clone or express the polynucleotide sequences of the invention.

The invention also encompasses the host cells transformed by a vector according to the invention. These cells may be obtained by introducing into host cells a nucleotide sequence inserted into a vector as defined above, and then culturing the said cells under conditions allowing the replication and/or the expression of the polynucleotide sequences of the subject invention.

The host cell may be chosen from eukaryotic or prokaryotic systems, such as for example bacterial cells, (Gram negative or Gram positive), yeast cells (for example, Saccharomyces cerevisiae or Pichia pastoris), animal cells (such as Chinese hamster ovary (CHO) cells), plant cells, and/or insect cells using baculovirus vectors. In some embodiments, the host cells for expression of the polypeptides include, and are not limited to, those taught in U.S. Pat. Nos. 6,319,691, 6,277,375, 5,643,570, or 5,565,335, each of which is incorporated by reference in its entirety, including all references cited within each respective patent.

Furthermore, a host cell strain may be chosen which modulates the expression of the inserted sequences, or modifies and processes the gene product in the specific fashion desired. Expression from certain promoters can be elevated in the presence of certain inducers; thus, expression of the genetically engineered polypeptide may be controlled. Furthermore, different host cells have characteristic and specific mechanisms for the translational and post-translational processing and modification (e.g., glycosylation, phosphorylation) of proteins. Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may effect processing reactions to different extents.

The subject invention also provides methods of identifying an individual at risk for liver failure comprising the detection of: a) a polynucleotide comprising SEQ ID NO: 1; or b) a polypeptide comprising SEQ ID NO: 2; in a biological sample obtained from said individual, wherein the presence of said polynucleotide or said polypeptide is associated with liver failure (or end stage liver failure). As discussed infra, the presence or absence of the polynucleotide or polypeptide can be determined using standard methodologies known in the art.

The subject invention further provides a method of classifying potential liver transplantation patients on a transplant list or in a liver transplant classification system that utilizes the presence or absence of a polynucleotide comprising SEQ ID NO: 1 or a polypeptide comprising SEQ ID NO: 2. In this aspect of the invention, the presence of a polynucleotide comprising SEQ ID NO: 1 or a polypeptide comprising SEQ ID NO: 2 is indicative of a patient that is very likely to experience complete liver failure. As such, it is important that such patients be given high priority in receiving a liver transplant prior to the complete failure of their livers.

Accordingly, the subject invention provides a method of creating, reordering or revising a classification system of liver transplant patients comprising: (a) analyzing a hepatic biological sample of a potential liver transplant patient for the presence or absence of a polynucleotide comprising SEQ ID NO: 1 or a polypeptide comprising SEQ ID NO: 2; (b) categorizing the potential liver transplant patient on the basis of the presence or absence or said polynucleotide or polypeptide in said hepatic biological sample; and (c) assigning a potential liver transplant patient a high priority on a liver transplantation list or a classification system of liver transplant patients if said polynucleotide or said polypeptide is present in the hepatic biological sample of said potential liver transplant patient or reordering or revising the position of said potential liver transplant patient in the classification system or on a transplantation list such that the patient is more likely to receive a liver transplant or that the priority of the patient on a liver transplantation list or in a classification system of liver transplant patients is increased if said polynucleotide or said polypeptide is present in the biological sample of said patient.

Also provided by the subject invention are methods of reducing the expression of the polypeptide of SEQ ID NO: 2 or the polynucleotide of SEQ ID NO: 1 comprising the administration of a polynucleotide that reduces the expression of SEQ ID NO: 1 or SEQ ID NO: 2 to a cell or individual. Expression of SEQ ID NOs: 1 and 2 can be reduced by RNA interference or antisense technologies.

RNAi is an efficient process whereby double-stranded RNA (dsRNA, also referred to herein as siRNAs or ds siRNAs, for double-stranded small interfering RNAs) induces the sequence-specific degradation of targeted mRNA in animal and plant cells (Hutvagner and Zamore, 2002); Sharp 2001). In mammalian cells, RNAi can be triggered by 21-nucleotide (nt) duplexes of small interfering RNA (siRNA) (Chiu et al., 2002; Elbashir et al., 2001), or by micro-RNAs (miRNA), functional small-hairpin RNA (shRNA), or other dsRNAs which can be expressed in vivo using DNA templates with RNA polymerase III promoters (Zeng et al., 2002; Paddison et al., 2002; Lee et al., 2002; Paul et al., 2002; Tuschl, T., 2002; Yu et al., 2002; McManus et al., 2002; Sui et al., 2002), each of which are incorporated herein by reference in their entirety.

The scientific literature is replete with reports of endogenous and exogenous gene expression silencing using siRNA, highlighting their therapeutic potential (Gupta, S. et al., 2004; Takaku, 2004; Pardridge, 2004; Zheng, 2004; Shen, 2004; Fuchs et al., 2004; Wadhwa et al., 2004; Ichim et al., 2004; Jana et al., 2004; Ryther et al., 2005; Chae et al, 2004; de Fougerolles et al., 2005), each of which is incorporated herein by reference in its entirety. Therapeutic silencing of endogenous genes by systemic administration of siRNAs has been described in the literature (Kim et al., 2004; Soutschek et al., 2004; Pardridge, 2004, each of which is incorporated herein by reference in its entirety.

Accordingly, the invention includes such interfering RNA molecules that are targeted to the SEQ ID NO: 1. The interfering RNA molecules are capable, when suitably introduced into or expressed within a cell that otherwise expresses SEQ ID NO: 1, of suppressing expression of SEQ ID NO: 1 by RNAi. The interfering RNA may be a double stranded siRNA. As the skilled person will appreciate, and as explained further herein, an siRNA molecule may include a short 3′ DNA sequence also. Alternatively, the nucleic acid may be a DNA (usually double-stranded DNA) which, when transcribed in a cell, yields an RNA having two complementary portions joined via a spacer, such that the RNA takes the form of a hairpin when the complementary portions hybridize with each other. In a mammalian cell, the hairpin structure may be cleaved from the molecule by the enzyme DICER, to yield two distinct, but hybridized, RNA molecules.

Reduction (suppression) of expression results in a decrease of the amounts of SEQ ID NO: 1 and SEQ ID NO: 2 within the cell Preferred degrees of suppression are at least 50%, 60%, 70%, 80%, 85%, or 90%. A level of suppression between 90% and 100% is generally considered a “silencing” of gene expression.

Another embodiment of the invention provides an interfering RNA that is generally targeted to the sequence of nucleotides that includes at least one of the nucleotides at positions 1095 through 1197 of SEQ ID NO: 1 or spans positions 1095 to 1197 of SEQ ID NO: 1. In a specific embodiment, interfering RNA polynucleotides comprise SEQ ID NOs: 3 or 4. By the term “generally targeted” it is intended that the polynucleotide targets a sequence that overlaps or is within about 10 to 100 nucleotides of positions 1095 through 1197 of SEQ ID NO: 1.

It is expected that perfect identity/complementarity between the interfering RNA of the invention and the target sequence, although preferred, is not essential. Accordingly, the interfering RNA may include a single mismatch compared to the mRNA of SEQ ID NO: 1 or the mRNA of SEQ ID NO: 1 (and wherein the interfering RNA includes a sequence of nucleotides that includes at least one of the nucleotides at positions 1095 through 1197 of SEQ ID NO: 1 or spans positions 1095 to 1197 of SEQ ID NO: 1) that spans positions 1095 through 1197 of SEQ ID NO: 1. However, the presence of even a single mismatch is likely to lead to reduced efficiency, thus, the absence of mismatches is preferred. When present, 3′ overhangs may be excluded from the consideration of the number of mismatches.

The term “complementarity” is not limited to conventional base pairing between nucleic acid consisting of naturally occurring ribo- and/or deoxyribonucleotides, but also includes base pairing between mRNA and nucleic acids of the invention that include non-natural nucleotides.

Short interfering RNAs (siRNAs) induce the sequence-specific suppression or silencing (i.e., reducing expression which may be to the extent of partial or complete inhibition) genes by the process of RNAi. Thus, siRNA is the intermediate effector molecule of the RNAi process. The nucleic acid molecules (polynucleotides) or constructs of the invention include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA of SEQ ID NO: 1 (typically a region including at least one nucleotide found at positions 1095 through 1197 of SEQ ID NO: 1 or spanning positions 1095 through 1197 of SEQ ID NO: 1) and the other strand is identical or substantially identical to the first strand. The dsRNA molecules of the invention can be chemically synthesized, or can be transcribed in vitro from a DNA template, or in vivo from, e.g., shRNA. The dsRNA molecules can be designed using any method known in the art, for instance, by using the following protocol:

1. Using any method known in the art, compare the potential targets to the appropriate genome database (human, mouse, rat, etc.) and eliminate from consideration any target sequences with significant homology to other coding sequences. One such method for sequence homology searches is known as BLAST, which is available at the National Center for Biotechnology Information (NCBI) web site of the National Institutes of Health. Also available on the NCBI webs site is the HomoloGene database, which is a publicly available system for automated detection of homologs among the annotated genes of several completely sequenced eukaryotic genomes and is readily utilized by those of ordinary skill in the art.

2. Select one or more sequences that meet the criteria for evaluation. Further general information regarding the design and use of siRNA can be found in “The siRNA User Guide,” available at the web site of the laboratory of Dr. Thomas Tuschl at Rockefeller University.

3. Negative control siRNAs preferably have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

Other computational tools that may be used to select siRNAs of the present invention include the Whitehead siRNA selection Web Server from the bioinformatics group at the Whitehead Institute for Biomedical Research in Cambridge, Mass., and other disclosed in Yuan et al. (2004) and Bonetta (2004), each of which are incorporated by reference herein in their entirety.

The polynucleotides of the invention can include both unmodified siRNAs and modified siRNAs as known in the art. Thus, the invention includes siRNA derivatives that include siRNA having two complementary strands of nucleic acid, such that the two strands are crosslinked. For example, a 3′ OH terminus of one of the strands can be modified, or the two strands can be crosslinked and modified at the 3′ OH terminus. The siRNA derivative can contain a single crosslink (e.g., a psoralen crosslink). In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way can improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acid compositions of the invention can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al. (2001) (describes nucleic acids loaded to polyalkylcyanoacrylate (PACA) nanoparticles); Fattal et al. (1998) (describes nucleic acids bound to nanoparticles); Schwab et al. (1994) (describes nucleic acids linked to intercalating agents, hydrophobic groups, polycations or PACA nanoparticles); and Godard et al. (1995) (describes nucleic acids linked to nanoparticles).

Because RNAi is believed to progress via at least one single stranded RNA intermediate, the skilled artisan will appreciate that ss-siRNAs (e.g., the antisense strand of a ds-siRNA) can also be designed as described herein and utilized according to the claimed methodologies.

There are a number of companies that will generate interfering RNAs for a specific gene. Thermo Electron Corporation has launched a custom synthesis service for synthetic short interfering RNA (siRNA). Each strand is composed of 18-20 RNA bases and two DNA bases overhang on the 3′ terminus. Dharmacon, Inc. provides siRNA duplexes using the 2′-ACE RNA synthesis technology. Qiagen uses TOM-chemistry to offer siRNA with individual coupling yields of over 99.5%.

Synthetic siRNAs can be delivered into cells by methods known in the art, including cationic liposome transfection and electroporation. However, these exogenous siRNA generally show short term persistence of the silencing effect (4 to 5 days in cultured cells), which may be beneficial in certain embodiments. To obtain longer term suppression of AS expression and to facilitate delivery under certain circumstances, one or more siRNA duplexes, e.g., AS ds siRNA, can be expressed within cells from recombinant DNA constructs. Such methods for expressing siRNA duplexes within cells from recombinant DNA constructs to allow longer-term target gene suppression in cells are known in the art, including mammalian Pol III promoter systems (e.g., H1 or U6/snRNA promoter systems (Tuschl 2002) capable of expressing functional double-stranded siRNAs; (Bagella et al., 1998; Lee et al., 2002; Miyagishi et al., 2002; Paul et al., 2002; Yu et al., 2002; Sui et al., 2002). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by an H1 or U6 snRNA promoter can be expressed in cells, and can inhibit target gene expression (Bagella et al., 1998; Lee et al., 2002; Miyagishi et al., 2002; Paul et al., 2002; Yu et al., 2002; Sui et al., 2002). Constructs containing siRNA sequence(s) under the control of a T7 promoter also make functional siRNAs when co-transfected into the cells with a vector expressing T7 RNA polymerase (Jacque 2002). A single construct may contain multiple sequences coding for siRNAs, such as multiple regions of SEQ ID NO: 1, providing that at least one of such sequences includes the region including at least one of the nucleotides at positions 1095 through 1197 of SEQ ID NO: 1 or spans positions 1095 to 1197 of SEQ ID NO: 1, and can be driven, for example, by separate PolIII promoter sites.

Animal cells express a range of noncoding RNAs of approximately 22 nucleotides termed micro RNA (miRNAs) which can regulate gene expression at the post transcriptional or translational level during animal development. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng, 2002). When expressed by DNA vectors containing polymerase III promoters, micro-RNA designed hairpins can silence gene expression (McManus, 2002). Viral-mediated delivery mechanisms can also be used to induce specific silencing of targeted genes through expression of siRNA, for example, by generating recombinant adenoviruses harboring siRNA under RNA Pol II promoter transcription control (Xia et al., 2002). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. In an animal model, whole-embryo electroporation can efficiently deliver synthetic siRNA into post-implantation mouse embryos (Calegari et al., 2002). In adult mice, efficient delivery of siRNA can be accomplished by the “high-pressure” delivery technique, a rapid injection (within 5 seconds) of a large volume of siRNA containing solution into animal via the tail vein (McCaffrey (2002); Lewis, 2002). Nanoparticles, liposomes and other cationic lipid molecules can also be used to deliver siRNA into animals. A gel-based agarose/liposome/siRNA formulation is also available (Jiamg M. et al., 2004).

Engineered RNA precursors, introduced into cells or whole organisms as described herein, will lead to the production of a desired siRNA molecule. Such an siRNA molecule will then associate with endogenous protein components of the RNAi pathway to bind to and target a specific mRNA sequence for cleavage and destruction. In this fashion, the mRNA to be targeted by the siRNA generated from the engineered RNA precursor will be depleted from the cell or organism, leading to a decrease in the concentration of any translational product encoded by that mRNA in the cell or organism. The RNA precursors are typically nucleic acid molecules that individually encode either one strand of a dsRNA or encode the entire nucleotide sequence of an RNA hairpin loop structure.

An “antisense” nucleic acid sequence (antisense oligonucleotide) can include a nucleotide sequence that is complementary to a “sense” nucleic acid encoding a protein, e.g., complementary to the coding strand of a double-stranded cDNA molecule or complementary to a target nucleotide region of SEQ ID NO: 1 that includes at least one of the nucleotides at positions 1095 through 1197 of SEQ ID NO: 1 or spans nucleotides 1095 through 1197 of SEQ ID NO: 1. Antisense nucleic acid sequences and delivery methods are well known in the art (Goodchild J., 2004; Clawson G. A. et al., 2004), which are incorporated herein by reference in their entirety. An antisense oligonucleotide can be, for example, about 7, 10, 15, 20, 25, 30, 35, 40, 45, 50, 55, 60, 65, 70, 75, 80, or more nucleotides in length. In one aspect of the invention, the antisense sequence spans nucleotides 1095 through 1197 of SEQ ID NO: 1. Other aspects of the invention provide antisense sequences that span any 7, 10, 15, 20, 25, 30, 35, 40, 45, 50, 55, 60, 65, 70, 75, 80, 85, 90, 95, or 100 consecutive nucleotides of the span of nucleotides comprising, or consisting of, nucleotides 1095-1197 of SEQ ID NO: 1. Another aspect of the invention comprises any span of nucleic acids set forth in Table 3 or 4 of this application.

An antisense nucleic acid sequence can be designed such that it is complementary to the entirety of SEQ ID NO: 1 or to only a portion of SEQ ID NO: 1. For example, the antisense oligonucleotide can be complementary to the region surrounding positions 1095 through 1197 of SEQ ID NO: 1, e.g., between the 10 nucleotides 5′ and 10 nucleotides 3′ to any one of nucleotides 1095 through 1188 of SEQ ID NO: 1. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., systemically or locally by direct injection at a tissue site (the liver)), or generated in situ such that they hybridize with or bind to cellular mRNA and/or genomic DNA encoding SEQ ID NO: 1 to thereby inhibit its expression. Alternatively, antisense nucleic acid molecules can be modified to target hepatic cells and then administered systemically. For systemic administration, antisense molecules can be modified such that they specifically bind to receptors or antigens expressed on a selected cell surface, e.g., by linking the antisense nucleic acid molecules to peptides or antibodies that bind to hepatic cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In yet another embodiment, the antisense oligonucleotide of the invention is an alpha-anomeric nucleic acid molecule. An alpha-anomeric nucleic acid molecule forms specific double-stranded hybrids with complementary RNA in which, contrary to the usual beta-units, the strands run parallel to each other (Gaultier et al., 1987). The antisense nucleic acid molecule can also comprise a 2′-o-methylribonucleotide (Inoue et al., 1987) or a chimeric RNA-DNA analogue (Inoue et al., 1987a).

All patents, patent applications, provisional applications, and publications referred to or cited herein are incorporated by reference in their entirety, including all figures and tables, to the extent they are not inconsistent with the explicit teachings of this specification.

TABLE 1
Fragments of SEQ ID NO: 2

| Fragment | Y is any integer | |
|---|---|---|
| Length | selected from | |
| (amino | between, and | |
| acids) | including: | Z |
| 5 | 1 and 388 | Y + 4 |
| 6 | 1 and 387 | Y + 5 |
| 7 | 1 and 386 | Y + 6 |
| 8 | 1 and 385 | Y + 7 |
| 9 | 1 and 384 | Y + 8 |
| 10 | 1 and 383 | Y + 9 |
| 11 | 1 and 382 | Y + 10 |
| 12 | 1 and 381 | Y + 11 |
| 13 | 1 and 380 | Y + 12 |
| 14 | 1 and 379 | Y + 13 |
| 15 | 1 and 378 | Y + 14 |
| 16 | 1 and 377 | Y + 15 |
| 17 | 1 and 376 | Y + 16 |
| 18 | 1 and 375 | Y + 17 |
| 19 | 1 and 374 | Y + 18 |
| 20 | 1 and 373 | Y + 19 |
| 21 | 1 and 372 | Y + 20 |
| 22 | 1 and 371 | Y + 21 |
| 23 | 1 and 370 | Y + 22 |
| 24 | 1 and 369 | Y + 23 |
| 25 | 1 and 368 | Y + 24 |
| 26 | 1 and 367 | Y + 25 |
| 27 | 1 and 366 | Y + 26 |
| 28 | 1 and 365 | Y + 27 |
| 29 | 1 and 364 | Y + 28 |
| 30 | 1 and 363 | Y + 29 |
| 31 | 1 and 362 | Y + 30 |
| 32 | 1 and 361 | Y + 31 |
| 33 | 1 and 360 | Y + 32 |
| 34 | 1 and 359 | Y + 33 |
| 35 | 1 and 358 | Y + 34 |
| 36 | 1 and 357 | Y + 35 |
| 37 | 1 and 356 | Y + 36 |
| 38 | 1 and 355 | Y + 37 |
| 39 | 1 and 354 | Y + 38 |
| 40 | 1 and 353 | Y + 39 |
| 41 | 1 and 352 | Y + 40 |
| 42 | 1 and 351 | Y + 41 |
| 43 | 1 and 350 | Y + 42 |
| 44 | 1 and 349 | Y + 43 |
| 45 | 1 and 348 | Y + 44 |
| 46 | 1 and 347 | Y + 45 |
| 47 | 1 and 346 | Y + 46 |
| 48 | 1 and 345 | Y + 47 |
| 49 | 1 and 344 | Y + 48 |
| 50 | 1 and 343 | Y + 49 |
| 51 | 1 and 342 | Y + 50 |
| 52 | 1 and 341 | Y + 51 |
| 53 | 1 and 340 | Y + 52 |
| 54 | 1 and 339 | Y + 53 |
| 55 | 1 and 338 | Y + 54 |
| 56 | 1 and 337 | Y + 55 |
| 57 | 1 and 336 | Y + 56 |
| 58 | 1 and 335 | Y + 57 |
| 59 | 1 and 334 | Y + 58 |
| 60 | 1 and 333 | Y + 59 |
| 61 | 1 and 332 | Y + 60 |
| 62 | 1 and 331 | Y + 61 |
| 63 | 1 and 330 | Y + 62 |
| 64 | 1 and 329 | Y + 63 |
| 65 | 1 and 328 | Y + 64 |
| 66 | 1 and 327 | Y + 65 |
| 67 | 1 and 326 | Y + 66 |
| 68 | 1 and 325 | Y + 67 |
| 69 | 1 and 324 | Y + 68 |
| 70 | 1 and 323 | Y + 69 |
| 71 | 1 and 322 | Y + 70 |
| 72 | 1 and 321 | Y + 71 |
| 73 | 1 and 320 | Y + 72 |
| 74 | 1 and 319 | Y + 73 |
| 75 | 1 and 318 | Y + 74 |
| 76 | 1 and 317 | Y + 75 |
| 77 | 1 and 316 | Y + 76 |
| 78 | 1 and 315 | Y + 77 |
| 79 | 1 and 314 | Y + 78 |
| 80 | 1 and 313 | Y + 79 |
| 81 | 1 and 312 | Y + 80 |
| 82 | 1 and 311 | Y + 81 |
| 83 | 1 and 310 | Y + 82 |
| 84 | 1 and 309 | Y + 83 |
| 85 | 1 and 308 | Y + 84 |
| 86 | 1 and 307 | Y + 85 |
| 87 | 1 and 306 | Y + 86 |
| 88 | 1 and 305 | Y + 87 |
| 89 | 1 and 304 | Y + 88 |
| 90 | 1 and 303 | Y + 89 |
| 91 | 1 and 302 | Y + 90 |
| 92 | 1 and 301 | Y + 91 |
| 93 | 1 and 300 | Y + 92 |
| 94 | 1 and 299 | Y + 93 |
| 95 | 1 and 298 | Y + 94 |
| 96 | 1 and 297 | Y + 95 |
| 97 | 1 and 296 | Y + 96 |
| 98 | 1 and 295 | Y + 97 |
| 99 | 1 and 294 | Y + 98 |
| 100 | 1 and 293 | Y + 99 |
| 101 | 1 and 292 | Y + 100 |
| 102 | 1 and 291 | Y + 101 |
| 103 | 1 and 290 | Y + 102 |
| 104 | 1 and 289 | Y + 103 |
| 105 | 1 and 288 | Y + 104 |
| 106 | 1 and 287 | Y + 105 |
| 107 | 1 and 286 | Y + 106 |
| 108 | 1 and 285 | Y + 107 |
| 109 | 1 and 284 | Y + 108 |
| 110 | 1 and 283 | Y + 109 |
| 111 | 1 and 282 | Y + 110 |
| 112 | 1 and 281 | Y + 111 |
| 113 | 1 and 280 | Y + 112 |
| 114 | 1 and 279 | Y + 113 |
| 115 | 1 and 278 | Y + 114 |
| 116 | 1 and 277 | Y + 115 |
| 117 | 1 and 276 | Y + 116 |
| 118 | 1 and 275 | Y + 117 |
| 119 | 1 and 274 | Y + 118 |
| 120 | 1 and 273 | Y + 119 |
| 121 | 1 and 272 | Y + 120 |
| 122 | 1 and 271 | Y + 121 |
| 123 | 1 and 270 | Y + 122 |
| 124 | 1 and 269 | Y + 123 |
| 125 | 1 and 268 | Y + 124 |
| 126 | 1 and 267 | Y + 125 |
| 127 | 1 and 266 | Y + 126 |
| 128 | 1 and 265 | Y + 127 |
| 129 | 1 and 264 | Y + 128 |
| 130 | 1 and 263 | Y + 129 |
| 131 | 1 and 262 | Y + 130 |
| 132 | 1 and 261 | Y + 131 |
| 133 | 1 and 260 | Y + 132 |
| 134 | 1 and 259 | Y + 133 |
| 135 | 1 and 258 | Y + 134 |
| 136 | 1 and 257 | Y + 135 |
| 137 | 1 and 256 | Y + 136 |
| 138 | 1 and 255 | Y + 137 |
| 139 | 1 and 254 | Y + 138 |
| 140 | 1 and 253 | Y + 139 |
| 141 | 1 and 252 | Y + 140 |
| 142 | 1 and 251 | Y + 141 |
| 143 | 1 and 250 | Y + 142 |
| 144 | 1 and 249 | Y + 143 |
| 145 | 1 and 248 | Y + 144 |
| 146 | 1 and 247 | Y + 145 |
| 147 | 1 and 246 | Y + 146 |
| 148 | 1 and 245 | Y + 147 |
| 149 | 1 and 244 | Y + 148 |
| 150 | 1 and 243 | Y + 149 |
| 151 | 1 and 242 | Y + 150 |
| 152 | 1 and 241 | Y + 151 |
| 153 | 1 and 240 | Y + 152 |
| 154 | 1 and 239 | Y + 153 |
| 155 | 1 and 238 | Y + 154 |
| 156 | 1 and 237 | Y + 155 |
| 157 | 1 and 236 | Y + 156 |
| 158 | 1 and 235 | Y + 157 |
| 159 | 1 and 234 | Y + 158 |
| 160 | 1 and 233 | Y + 159 |
| 161 | 1 and 232 | Y + 160 |
| 162 | 1 and 231 | Y + 161 |
| 163 | 1 and 230 | Y + 162 |
| 164 | 1 and 229 | Y + 163 |
| 165 | 1 and 228 | Y + 164 |
| 166 | 1 and 227 | Y + 165 |
| 167 | 1 and 226 | Y + 166 |
| 168 | 1 and 225 | Y + 167 |
| 169 | 1 and 224 | Y + 168 |
| 170 | 1 and 223 | Y + 169 |
| 171 | 1 and 222 | Y + 170 |
| 172 | 1 and 221 | Y + 171 |
| 173 | 1 and 220 | Y + 172 |
| 174 | 1 and 219 | Y + 173 |
| 175 | 1 and 218 | Y + 174 |
| 176 | 1 and 217 | Y + 175 |
| 177 | 1 and 216 | Y + 176 |
| 178 | 1 and 215 | Y + 177 |
| 179 | 1 and 214 | Y + 178 |
| 180 | 1 and 213 | Y + 179 |
| 181 | 1 and 212 | Y + 180 |
| 182 | 1 and 211 | Y + 181 |
| 183 | 1 and 210 | Y + 182 |
| 184 | 1 and 209 | Y + 183 |
| 185 | 1 and 208 | Y + 184 |
| 186 | 1 and 207 | Y + 185 |
| 187 | 1 and 206 | Y + 186 |
| 188 | 1 and 205 | Y + 187 |
| 189 | 1 and 204 | Y + 188 |
| 190 | 1 and 203 | Y + 189 |
| 191 | 1 and 202 | Y + 190 |
| 192 | 1 and 201 | Y + 191 |
| 193 | 1 and 200 | Y + 192 |
| 194 | 1 and 199 | Y + 193 |
| 195 | 1 and 198 | Y + 194 |
| 196 | 1 and 197 | Y + 195 |
| 197 | 1 and 196 | Y + 196 |
| 198 | 1 and 195 | Y + 197 |
| 199 | 1 and 194 | Y + 198 |
| 200 | 1 and 193 | Y + 199 |
| 201 | 1 and 192 | Y + 200 |
| 202 | 1 and 191 | Y + 201 |
| 203 | 1 and 190 | Y + 202 |
| 204 | 1 and 189 | Y + 203 |
| 205 | 1 and 188 | Y + 204 |
| 206 | 1 and 187 | Y + 205 |
| 207 | 1 and 186 | Y + 206 |
| 208 | 1 and 185 | Y + 207 |
| 209 | 1 and 184 | Y + 208 |
| 210 | 1 and 183 | Y + 209 |
| 211 | 1 and 182 | Y + 210 |
| 212 | 1 and 181 | Y + 211 |
| 213 | 1 and 180 | Y + 212 |
| 214 | 1 and 179 | Y + 213 |
| 215 | 1 and 178 | Y + 214 |
| 216 | 1 and 177 | Y + 215 |
| 217 | 1 and 176 | Y + 216 |
| 218 | 1 and 175 | Y + 217 |
| 219 | 1 and 174 | Y + 218 |
| 220 | 1 and 173 | Y + 219 |
| 221 | 1 and 172 | Y + 220 |
| 222 | 1 and 171 | Y + 221 |
| 223 | 1 and 170 | Y + 222 |
| 224 | 1 and 169 | Y + 223 |
| 225 | 1 and 168 | Y + 224 |
| 226 | 1 and 167 | Y + 225 |
| 227 | 1 and 166 | Y + 226 |
| 228 | 1 and 165 | Y + 227 |
| 229 | 1 and 164 | Y + 228 |
| 230 | 1 and 163 | Y + 229 |
| 231 | 1 and 162 | Y + 230 |
| 232 | 1 and 161 | Y + 231 |
| 233 | 1 and 160 | Y + 232 |
| 234 | 1 and 159 | Y + 233 |
| 235 | 1 and 158 | Y + 234 |
| 236 | 1 and 157 | Y + 235 |
| 237 | 1 and 156 | Y + 236 |
| 238 | 1 and 155 | Y + 237 |
| 239 | 1 and 154 | Y + 238 |
| 240 | 1 and 153 | Y + 239 |
| 241 | 1 and 152 | Y + 240 |
| 242 | 1 and 151 | Y + 241 |
| 243 | 1 and 150 | Y + 242 |
| 244 | 1 and 149 | Y + 243 |
| 245 | 1 and 148 | Y + 244 |
| 246 | 1 and 147 | Y + 245 |
| 247 | 1 and 146 | Y + 246 |
| 248 | 1 and 145 | Y + 247 |
| 249 | 1 and 144 | Y + 248 |
| 250 | 1 and 143 | Y + 249 |
| 251 | 1 and 142 | Y + 250 |
| 252 | 1 and 141 | Y + 251 |
| 253 | 1 and 140 | Y + 252 |
| 254 | 1 and 139 | Y + 253 |
| 255 | 1 and 138 | Y + 254 |
| 256 | 1 and 137 | Y + 255 |
| 257 | 1 and 136 | Y + 256 |
| 258 | 1 and 135 | Y + 257 |
| 259 | 1 and 134 | Y + 258 |
| 260 | 1 and 133 | Y + 259 |
| 261 | 1 and 132 | Y + 260 |
| 262 | 1 and 131 | Y + 261 |
| 263 | 1 and 130 | Y + 262 |
| 264 | 1 and 129 | Y + 263 |
| 265 | 1 and 128 | Y + 264 |
| 266 | 1 and 127 | Y + 265 |
| 267 | 1 and 126 | Y + 266 |
| 268 | 1 and 125 | Y + 267 |
| 269 | 1 and 124 | Y + 268 |
| 270 | 1 and 123 | Y + 269 |
| 271 | 1 and 122 | Y + 270 |
| 272 | 1 and 121 | Y + 271 |
| 273 | 1 and 120 | Y + 272 |
| 274 | 1 and 119 | Y + 273 |
| 275 | 1 and 118 | Y + 274 |
| 276 | 1 and 117 | Y + 275 |
| 277 | 1 and 116 | Y + 276 |
| 278 | 1 and 115 | Y + 277 |
| 279 | 1 and 114 | Y + 278 |
| 280 | 1 and 113 | Y + 279 |
| 281 | 1 and 112 | Y + 280 |
| 282 | 1 and 111 | Y + 281 |
| 283 | 1 and 110 | Y + 282 |
| 284 | 1 and 109 | Y + 283 |
| 285 | 1 and 108 | Y + 284 |
| 286 | 1 and 107 | Y + 285 |
| 287 | 1 and 106 | Y + 286 |
| 288 | 1 and 105 | Y + 287 |
| 289 | 1 and 104 | Y + 288 |
| 290 | 1 and 103 | Y + 289 |
| 291 | 1 and 102 | Y + 290 |
| 292 | 1 and 101 | Y + 291 |
| 293 | 1 and 100 | Y + 292 |
| 294 | 1 and 99 | Y + 293 |
| 295 | 1 and 98 | Y + 294 |
| 296 | 1 and 97 | Y + 295 |
| 297 | 1 and 96 | Y + 296 |
| 298 | 1 and 95 | Y + 297 |
| 299 | 1 and 94 | Y + 298 |
| 300 | 1 and 93 | Y + 299 |
| 301 | 1 and 92 | Y + 300 |
| 302 | 1 and 91 | Y + 301 |
| 303 | 1 and 90 | Y + 302 |
| 304 | 1 and 89 | Y + 303 |
| 305 | 1 and 88 | Y + 304 |
| 306 | 1 and 87 | Y + 305 |
| 307 | 1 and 86 | Y + 306 |
| 308 | 1 and 85 | Y + 307 |
| 309 | 1 and 84 | Y + 308 |
| 310 | 1 and 83 | Y + 309 |
| 311 | 1 and 82 | Y + 310 |
| 312 | 1 and 81 | Y + 311 |
| 313 | 1 and 80 | Y + 312 |
| 314 | 1 and 79 | Y + 313 |
| 315 | 1 and 78 | Y + 314 |
| 316 | 1 and 77 | Y + 315 |
| 317 | 1 and 76 | Y + 316 |
| 318 | 1 and 75 | Y + 317 |
| 319 | 1 and 74 | Y + 318 |
| 320 | 1 and 73 | Y + 319 |
| 321 | 1 and 72 | Y + 320 |
| 322 | 1 and 71 | Y + 321 |
| 323 | 1 and 70 | Y + 322 |
| 324 | 1 and 69 | Y + 323 |
| 325 | 1 and 68 | Y + 324 |
| 326 | 1 and 67 | Y + 325 |
| 327 | 1 and 66 | Y + 326 |
| 328 | 1 and 65 | Y + 327 |
| 329 | 1 and 64 | Y + 328 |
| 330 | 1 and 63 | Y + 329 |
| 331 | 1 and 62 | Y + 330 |
| 332 | 1 and 61 | Y + 331 |
| 333 | 1 and 60 | Y + 332 |
| 334 | 1 and 59 | Y + 333 |
| 335 | 1 and 58 | Y + 334 |
| 336 | 1 and 57 | Y + 335 |
| 337 | 1 and 56 | Y + 336 |
| 338 | 1 and 55 | Y + 337 |
| 339 | 1 and 54 | Y + 338 |
| 340 | 1 and 53 | Y + 339 |
| 341 | 1 and 52 | Y + 340 |
| 342 | 1 and 51 | Y + 341 |
| 343 | 1 and 50 | Y + 342 |
| 344 | 1 and 49 | Y + 343 |
| 345 | 1 and 48 | Y + 344 |
| 346 | 1 and 47 | Y + 345 |
| 347 | 1 and 46 | Y + 346 |
| 348 | 1 and 45 | Y + 347 |
| 349 | 1 and 44 | Y + 348 |
| 350 | 1 and 43 | Y + 349 |
| 351 | 1 and 42 | Y + 350 |
| 352 | 1 and 41 | Y + 351 |
| 353 | 1 and 40 | Y + 352 |
| 354 | 1 and 39 | Y + 353 |
| 355 | 1 and 38 | Y + 354 |
| 356 | 1 and 37 | Y + 355 |
| 357 | 1 and 36 | Y + 356 |
| 358 | 1 and 35 | Y + 357 |
| 359 | 1 and 34 | Y + 358 |
| 360 | 1 and 33 | Y + 359 |
| 361 | 1 and 32 | Y + 360 |
| 362 | 1 and 31 | Y + 361 |
| 363 | 1 and 30 | Y + 362 |
| 364 | 1 and 29 | Y + 363 |
| 365 | 1 and 28 | Y + 364 |
| 366 | 1 and 27 | Y + 365 |
| 367 | 1 and 26 | Y + 366 |
| 368 | 1 and 25 | Y + 367 |
| 369 | 1 and 24 | Y + 368 |
| 370 | 1 and 23 | Y + 369 |
| 371 | 1 and 22 | Y + 370 |
| 372 | 1 and 21 | Y + 371 |
| 373 | 1 and 20 | Y + 372 |
| 374 | 1 and 19 | Y + 373 |
| 375 | 1 and 18 | Y + 374 |
| 376 | 1 and 17 | Y + 375 |
| 377 | 1 and 16 | Y + 376 |
| 378 | 1 and 15 | Y + 377 |
| 379 | 1 and 14 | Y + 378 |
| 380 | 1 and 13 | Y + 379 |
| 381 | 1 and 12 | Y + 380 |
| 382 | 1 and 11 | Y + 381 |
| 383 | 1 and 10 | Y + 382 |
| 384 | 1 and 9 | Y + 383 |
| 385 | 1 and 8 | Y + 384 |
| 386 | 1 and 7 | Y + 385 |
| 387 | 1 and 6 | Y + 386 |
| 388 | 1 and 5 | Y + 387 |
| 389 | 1 and 4 | Y + 388 |
| 390 | 1 and 3 | Y + 389 |
| 391 | 1 and 2 | Y + 390 |

TABLE 2
Percent Identity

91.00
91.01
91.02
91.03
91.04
91.05
91.06
91.07
91.08
91.09
91.10
91.11
91.12
91.13
91.14
91.15
91.16
91.17
91.18
91.19
91.20
91.21
91.22
91.23
91.24
91.25
91.26
91.27
91.28
91.29
91.30
91.31
91.32
91.33
91.34
91.35
91.36
91.37
91.38
91.39
91.40
91.41
91.42
91.43
91.44
91.45
91.46
91.47
91.48
91.49
91.50
91.51
91.52
91.53
91.54
91.55
91.56
91.57
91.58
91.59
91.60
91.61
91.62
91.63
91.64
91.65
91.66
91.67
91.68
91.69
91.70
91.71
91.72
91.73
91.74
91.75
91.76
91.77
91.78
91.79
91.80
91.81
91.82
91.83
91.84
91.85
91.86
91.87
91.88
91.89
91.90
91.91
91.92
91.93
91.94
91.95
91.96
91.97
91.98
91.99
92.00
92.01
92.02
92.03
92.04
92.05
92.06
92.07
92.08
92.09
92.10
92.11
92.12
92.13
92.14
92.15
92.16
92.17
92.18
92.19
92.20
92.21
92.22
92.23
92.24
92.25
92.26
92.27
92.28
92.29
92.30
92.31
92.32
92.33
92.34
92.35
92.36
92.37
92.38
92.39
92.40
92.41
92.42
92.43
92.44
92.45
92.46
92.47
92.48
92.49
92.50
92.51
92.52
92.53
92.54
92.55
92.56
92.57
92.58
92.59
92.60
92.61
92.62
92.63
92.64
92.65
92.66
92.67
92.68
92.69
92.70
92.71
92.72
92.73
92.74
92.75
92.76
92.77
92.78
92.79
92.80
92.81
92.82
92.83
92.84
92.85
92.86
92.87
92.88
92.89
92.90
92.91
92.92
92.93
92.94
92.95
92.96
92.97
92.98
92.99
93.00
93.01
93.02
93.03
93.04
93.05
93.06
93.07
93.08
93.09
93.10
93.11
93.12
93.13
93.14
93.15
93.16
93.17
93.18
93.19
93.20
93.21
93.22
93.23
93.24
93.25
93.26
93.27
93.28
93.29
93.30
93.31
93.32
93.33
93.34
93.35
93.36
93.37
93.38
93.39
93.40
93.41
93.42
93.43
93.44
93.45
93.46
93.47
93.48
93.49
93.50
93.51
93.52
93.53
93.54
93.55
93.56
93.57
93.58
93.59
93.60
93.61
93.62
93.63
93.64
93.65
93.66
93.67
93.68
93.69
93.70
93.71
93.72
93.73
93.74
93.75
93.76
93.77
93.78
93.79
93.80
93.81
93.82
93.83
93.84
93.85
93.86
93.87
93.88
93.89
93.90
93.91
93.92
93.93
93.94
93.95
93.96
93.97
93.98
93.99
94.00
94.01
94.02
94.03
94.04
94.05
94.06
94.07
94.08
94.09
94.10
94.11
94.12
94.13
94.14
94.15
94.16
94.17
94.18
94.19
94.20
94.21
94.22
94.23
94.24
94.25
94.26
94.27
94.28
94.29
94.30
94.31
94.32
94.33
94.34
94.35
94.36
94.37
94.38
94.39
94.40
94.41
94.42
94.43
94.44
94.45
94.46
94.47
94.48
94.49
94.50
94.51
94.52
94.53
94.54
94.55
94.56
94.57
94.58
94.59
94.60
94.61
94.62
94.63
94.64
94.65
94.66
94.67
94.68
94.69
94.70
94.71
94.72
94.73
94.74
94.75
94.76
94.77
94.78
94.79
94.80
94.81
94.82
94.83
94.84
94.85
94.86
94.87
94.88
94.89
94.90
94.91
94.92
94.93
94.94
94.95
94.96
94.97
94.98
94.99
95.00
95.01
95.02
95.03
95.04
95.05
95.06
95.07
95.08
95.09
95.10
95.11
95.12
95.13
95.14
95.15
95.16
95.17
95.18
95.19
95.20
95.21
95.22
95.23
95.24
95.25
95.26
95.27
95.28
95.29
95.30
95.31
95.32
95.33
95.34
95.35
95.36
95.37
95.38
95.39
95.40
95.41
95.42
95.43
95.44
95.45
95.46
95.47
95.48
95.49
95.50
95.51
95.52
95.53
95.54
95.55
95.56
95.57
95.58
95.59
95.60
95.61
95.62
95.63
95.64
95.65
95.66
95.67
95.68
95.69
95.70
95.71
95.72
95.73
95.74
95.75
95.76
95.77
95.78
95.79
95.80
95.81
95.82
95.83
95.84
95.85
95.86
95.87
95.88
95.89
95.90
95.91
95.92
95.93
95.94
95.95
95.96
95.97
95.98
95.99
96.00
96.01
96.02
96.03
96.04
96.05
96.06
96.07
96.08
96.09
96.10
96.11
96.12
96.13
96.14
96.15
96.16
96.17
96.18
96.19
96.20
96.21
96.22
96.23
96.24
96.25
96.26
96.27
96.28
96.29
96.30
96.31
96.32
96.33
96.34
96.35
96.36
96.37
96.38
96.39
96.40
96.41
96.42
96.43
96.44
96.45
96.46
96.47
96.48
96.49
96.50
96.51
96.52
96.53
96.54
96.55
96.56
96.57
96.58
96.59
96.60
96.61
96.62
96.63
96.64
96.65
96.66
96.67
96.68
96.69
96.70
96.71
96.72
96.73
96.74
96.75
96.76
96.77
96.78
96.79
96.80
96.81
96.82
96.83
96.84
96.85
96.86
96.87
96.88
96.89
96.90
96.91
96.92
96.93
96.94
96.95
96.96
96.97
96.98
96.99
97.00
97.01
97.02
97.03
97.04
97.05
97.06
97.07
97.08
97.09
97.10
97.11
97.12
97.13
97.14
97.15
97.16
97.17
97.18
97.19
97.20
97.21
97.22
97.23
97.24
97.25
97.26
97.27
97.28
97.29
97.30
97.31
97.32
97.33
97.34
97.35
97.36
97.37
97.38
97.39
97.40
97.41
97.42
97.43
97.44
97.45
97.46
97.47
97.48
97.49
97.50
97.51
97.52
97.53
97.54
97.55
97.56
97.57
97.58
97.59
97.60
97.61
97.62
97.63
97.64
97.65
97.66
97.67
97.68
97.69
97.70
97.71
97.72
97.73
97.74
97.75
97.76
97.77
97.78
97.79
97.80
97.81
97.82
97.83
97.84
97.85
97.86
97.87
97.88
97.89
97.90
97.91
97.92
97.93
97.94
97.95
97.96
97.97
97.98
97.99
98.00
98.01
98.02
98.03
98.04
98.05
98.06
98.07
98.08
98.09
98.10
98.11
98.12
98.13
98.14
98.15
98.16
98.17
98.18
98.19
98.20
98.21
98.22
98.23
98.24
98.25
98.26
98.27
98.28
98.29
98.30
98.31
98.32
98.33
98.34
98.35
98.36
98.37
98.38
98.39
98.40
98.41
98.42
98.43
98.44
98.45
98.46
98.47
98.48
98.49
98.50
98.51
98.52
98.53
98.54
98.55
98.56
98.57
98.58
98.59
98.60
98.61
98.62
98.63
98.64
98.65
98.66
98.67
98.68
98.69
98.70
98.71
98.72
98.73
98.74
98.75
98.76
98.77
98.78
98.79
98.80
98.81
98.82
98.83
98.84
98.85
98.86
98.87
98.88
98.89
98.90
98.91
98.92
98.93
98.94
98.95
98.96
98.97
98.98
98.99
99.00
99.01
99.02
99.03
99.04
99.05
99.06
99.07
99.08
99.09
99.10
99.11
99.12
99.13
99.14
99.15
99.16
99.17
99.18
99.19
99.20
99.21
99.22
99.23
99.24
99.25
99.26
99.27
99.28
99.29
99.30
99.31
99.32
99.33
99.34
99.35
99.36
99.37
99.38
99.39
99.40
99.41
99.42
99.43
99.44
99.45
99.46
99.47
99.48
99.49
99.50
99.51
99.52
99.53
99.54
99.55
99.56
99.57
99.58
99.59
99.60
99.61
99.62
99.63
99.64
99.65
99.66
99.67
99.68
99.69
99.70
99.71
99.72
99.73
99.74
99.75
99.76
99.77
99.78
99.79
99.80
99.81
99.82
99.83
99.84
99.85
99.86
99.87
99.88
99.89
99.90
99.91
99.92
99.93
99.94
99.95
99.96
99.97
99.98
99.99
100.00

TABLE 3
Fragments of SEQ ID NO: 1
(spanning positions 1095-1197 of SEQ ID NO: 1)

| | Y is any integer | |
|---|---|---|
| Fragment | selected from | |
| Length | between, and | |
| (nucleotides) | including: | Z |
| 7 | 1095 and 1191 | Y + 6 |
| 8 | 1095 and 1190 | Y + 7 |
| 9 | 1095 and 1189 | Y + 8 |
| 10 | 1095 and 1188 | Y + 9 |
| 11 | 1095 and 1187 | Y + 10 |
| 12 | 1095 and 1186 | Y + 11 |
| 13 | 1095 and 1185 | Y + 12 |
| 14 | 1095 and 1184 | Y + 13 |
| 15 | 1095 and 1183 | Y + 14 |
| 16 | 1095 and 1182 | Y + 15 |
| 17 | 1095 and 1181 | Y + 16 |
| 18 | 1095 and 1180 | Y + 17 |
| 19 | 1095 and 1179 | Y + 18 |
| 20 | 1095 and 1178 | Y + 19 |
| 21 | 1095 and 1177 | Y + 20 |
| 22 | 1095 and 1176 | Y + 21 |
| 23 | 1095 and 1175 | Y + 22 |
| 24 | 1095 and 1174 | Y + 23 |
| 25 | 1095 and 1173 | Y + 24 |
| 26 | 1095 and 1172 | Y + 25 |
| 27 | 1095 and 1171 | Y + 26 |
| 28 | 1095 and 1170 | Y + 27 |
| 29 | 1095 and 1169 | Y + 28 |
| 30 | 1095 and 1168 | Y + 29 |
| 31 | 1095 and 1167 | Y + 30 |
| 32 | 1095 and 1166 | Y + 31 |
| 33 | 1095 and 1165 | Y + 32 |
| 34 | 1095 and 1164 | Y + 33 |
| 35 | 1095 and 1163 | Y + 34 |
| 36 | 1095 and 1162 | Y + 35 |
| 37 | 1095 and 1161 | Y + 36 |
| 38 | 1095 and 1160 | Y + 37 |
| 39 | 1095 and 1159 | Y + 38 |
| 40 | 1095 and 1158 | Y + 39 |
| 41 | 1095 and 1157 | Y + 40 |
| 42 | 1095 and 1156 | Y + 41 |
| 43 | 1095 and 1155 | Y + 42 |
| 44 | 1095 and 1154 | Y + 43 |
| 45 | 1095 and 1153 | Y + 44 |
| 46 | 1095 and 1152 | Y + 45 |
| 47 | 1095 and 1151 | Y + 46 |
| 48 | 1095 and 1150 | Y + 47 |
| 49 | 1095 and 1149 | Y + 48 |
| 50 | 1095 and 1148 | Y + 49 |
| 51 | 1095 and 1147 | Y + 50 |
| 52 | 1095 and 1146 | Y + 51 |
| 53 | 1095 and 1145 | Y + 52 |
| 54 | 1095 and 1144 | Y + 53 |
| 55 | 1095 and 1143 | Y + 52 |
| 56 | 1095 and 1142 | Y + 53 |
| 57 | 1095 and 1141 | Y + 54 |
| 58 | 1095 and 1140 | Y + 55 |
| 59 | 1095 and 1139 | Y + 56 |
| 60 | 1095 and 1138 | Y + 57 |
| 61 | 1095 and 1137 | Y + 58 |
| 62 | 1095 and 1136 | Y + 59 |
| 63 | 1095 and 1135 | Y + 60 |
| 64 | 1095 and 1134 | Y + 61 |
| 65 | 1095 and 1133 | Y + 62 |
| 66 | 1095 and 1132 | Y + 63 |
| 67 | 1095 and 1131 | Y + 64 |
| 68 | 1095 and 1130 | Y + 65 |
| 69 | 1095 and 1129 | Y + 66 |
| 70 | 1095 and 1128 | Y + 67 |
| 71 | 1095 and 1127 | Y + 68 |
| 72 | 1095 and 1126 | Y + 69 |
| 73 | 1095 and 1125 | Y + 70 |
| 74 | 1095 and 1124 | Y + 71 |
| 75 | 1095 and 1123 | Y + 72 |
| 76 | 1095 and 1122 | Y + 73 |
| 77 | 1095 and 1121 | Y + 74 |
| 78 | 1095 and 1120 | Y + 75 |
| 79 | 1095 and 1119 | Y + 76 |
| 80 | 1095 and 1118 | Y + 77 |
| 81 | 1095 and 1117 | Y + 78 |
| 82 | 1095 and 1116 | Y + 79 |
| 83 | 1095 and 1115 | Y + 80 |
| 84 | 1095 and 1114 | Y + 81 |
| 85 | 1095 and 1113 | Y + 82 |
| 86 | 1095 and 1112 | Y + 83 |
| 87 | 1095 and 1111 | Y + 84 |
| 88 | 1095 and 1110 | Y + 85 |
| 89 | 1095 and 1109 | Y + 86 |
| 90 | 1095 and 1108 | Y + 87 |
| 91 | 1095 and 1107 | Y + 88 |
| 92 | 1095 and 1106 | Y + 89 |
| 93 | 1095 and 1105 | Y + 90 |
| 94 | 1095 and 1104 | Y + 91 |
| 95 | 1095 and 1103 | Y + 92 |
| 96 | 1095 and 1102 | Y + 93 |
| 97 | 1095 and 1101 | Y + 94 |
| 98 | 1095 and 1100 | Y + 95 |
| 99 | 1095 and 1099 | Y + 96 |
| 100 | 1095 and 1098 | Y + 97 |
| 101 | 1095 and 1097 | Y + 98 |
| 102 | 1095 and 1096 | Y + 99 |

TABLE 4
Fragments of SEQ ID NO: 1
(spanning positions 997-1197 of SEQ ID NO: 1)

| | Y is any integer | |
|---|---|---|
| Fragment | selected from | |
| Length | between, and | |
| (nucleotides) | including: | Z |
| 100 | 997 and 1098 | Y + 99 |
| 101 | 997 and 1097 | Y + 100 |
| 102 | 997 and 1096 | Y + 101 |
| 103 | 997 and 1095 | Y + 102 |
| 104 | 997 and 1094 | Y + 103 |
| 105 | 997 and 1093 | Y + 104 |
| 106 | 997 and 1092 | Y + 105 |
| 107 | 997 and 1091 | Y + 106 |
| 108 | 997 and 1090 | Y + 107 |
| 109 | 997 and 1089 | Y + 108 |
| 110 | 997 and 1088 | Y + 109 |
| 111 | 997 and 1087 | Y + 110 |
| 112 | 997 and 1086 | Y + 111 |
| 113 | 997 and 1085 | Y + 112 |
| 114 | 997 and 1084 | Y + 113 |
| 115 | 997 and 1083 | Y + 114 |
| 116 | 997 and 1082 | Y + 115 |
| 117 | 997 and 1081 | Y + 116 |
| 118 | 997 and 1080 | Y + 117 |
| 119 | 997 and 1079 | Y + 118 |
| 120 | 997 and 1078 | Y + 119 |
| 121 | 997 and 1077 | Y + 120 |
| 122 | 997 and 1076 | Y + 121 |
| 123 | 997 and 1075 | Y + 122 |
| 124 | 997 and 1074 | Y + 123 |
| 125 | 997 and 1073 | Y + 124 |
| 126 | 997 and 1072 | Y + 125 |
| 127 | 997 and 1071 | Y + 126 |
| 128 | 997 and 1070 | Y + 127 |
| 129 | 997 and 1069 | Y + 128 |
| 130 | 997 and 1068 | Y + 129 |
| 131 | 997 and 1067 | Y + 130 |
| 132 | 997 and 1066 | Y + 131 |
| 133 | 997 and 1065 | Y + 132 |
| 134 | 997 and 1064 | Y + 133 |
| 135 | 997 and 1063 | Y + 134 |
| 136 | 997 and 1062 | Y + 135 |
| 137 | 997 and 1061 | Y + 136 |
| 138 | 997 and 1060 | Y + 137 |
| 139 | 997 and 1059 | Y + 138 |
| 140 | 997 and 1058 | Y + 139 |
| 141 | 997 and 1057 | Y + 140 |
| 142 | 997 and 1056 | Y + 141 |
| 143 | 997 and 1055 | Y + 142 |
| 144 | 997 and 1054 | Y + 143 |
| 145 | 997 and 1053 | Y + 144 |
| 146 | 997 and 1052 | Y + 145 |
| 147 | 997 and 1051 | Y + 146 |
| 148 | 997 and 1050 | Y + 147 |
| 149 | 997 and 1049 | Y + 148 |
| 150 | 997 and 1048 | Y + 149 |
| 151 | 997 and 1047 | Y + 150 |
| 152 | 997 and 1046 | Y + 151 |
| 153 | 997 and 1045 | Y + 152 |
| 154 | 997 and 1044 | Y + 153 |
| 155 | 997 and 1043 | Y + 154 |
| 156 | 997 and 1042 | Y + 155 |
| 157 | 997 and 1041 | Y + 156 |
| 158 | 997 and 1040 | Y + 157 |
| 159 | 997 and 1039 | Y + 158 |
| 160 | 997 and 1038 | Y + 159 |
| 161 | 997 and 1037 | Y + 160 |
| 162 | 997 and 1036 | Y + 161 |
| 163 | 997 and 1035 | Y + 162 |
| 164 | 997 and 1034 | Y + 163 |
| 165 | 997 and 1033 | Y + 164 |
| 166 | 997 and 1032 | Y + 165 |
| 167 | 997 and 1031 | Y + 166 |
| 168 | 997 and 1030 | Y + 167 |
| 169 | 997 and 1029 | Y + 168 |
| 170 | 997 and 1028 | Y + 169 |
| 171 | 997 and 1027 | Y + 170 |
| 172 | 997 and 1026 | Y + 171 |
| 173 | 997 and 1025 | Y + 172 |
| 174 | 997 and 1024 | Y + 173 |
| 175 | 997 and 1023 | Y + 174 |
| 176 | 997 and 1022 | Y + 175 |
| 177 | 997 and 1021 | Y + 176 |
| 178 | 997 and 1020 | Y + 177 |
| 179 | 997 and 1019 | Y + 178 |
| 180 | 997 and 1018 | Y + 179 |
| 181 | 997 and 1017 | Y + 180 |
| 182 | 997 and 1016 | Y + 181 |
| 183 | 997 and 1015 | Y + 182 |
| 184 | 997 and 1014 | Y + 183 |
| 185 | 997 and 1013 | Y + 184 |
| 186 | 997 and 1012 | Y + 185 |
| 187 | 997 and 1011 | Y + 186 |
| 188 | 997 and 1010 | Y + 187 |
| 189 | 997 and 1009 | Y + 188 |
| 190 | 997 and 1008 | Y + 189 |
| 191 | 997 and 1007 | Y + 190 |
| 192 | 997 and 1006 | Y + 191 |
| 193 | 997 and 1005 | Y + 192 |
| 194 | 997 and 1004 | Y + 193 |
| 195 | 997 and 1003 | Y + 194 |
| 196 | 997 and 1002 | Y + 195 |
| 197 | 997 and 1001 | Y + 196 |
| 198 | 997 and 1000 | Y + 197 |
| 199 | 997 and 999 | Y + 198 |
| 200 | 997 and 998 | Y + 199 |
| 201 | 997 | Y + 200 |

### REFERENCES

Altendorf et al. (1999-WWW, 2000) “Structure and Function of the Fₒ Complex of the ATP Synthase from Escherichia Coli” J. of Experimental Biology 203:19-28.
Altschul, S. F. et al. (1990) “Basic Local Alignment Search Tool” J. Mol. Biol. 215(3):403-410.
Alwine, J. C. et al. (1977) “Method for detection of specific RNAs in agarose gels by transfer to diazobenzyloxymethyl-paper and hybridization with DNA probes” Proc. Natl. Acad. Sci. 74:5350-5354.
Ausubel, M. et al. (1989) Current Protocols in Molecular Biology, Green Publishing Associates and Wiley Interscience, N.Y.
Baneyx, F. (1999) “Recombinant Protein Expression in Escherichia coli” Biotechnology 10:411-21.
Beltz, G. et al. (1983) “Isolation of multigene families and determination of homologies by filter hybridization methods” Methods of Enzymology, R. Wu, L. Grossman and K. Moldave [eds.] Academic Press, New York 100:266-285.
Benoist, C., Chambon, P. (1981) “In vivo sequence requirements of the SV40 early promoter region” Nature 290:304-310.
Berchtold, M. W. (1989) “A simple method for direct cloning and sequencing cDNA by the use of a single specific oligonucleotide and oligo(dT) in a polymerase chain reaction (PCR)” Nuc. Acids. Res. 17:453.
Bianchi, N. et al. (1997) “Biosensor technology and surface plasmon resonance for real-time detection of HIV-1 genomic sequences amplified by polymerase chain reaction” Clin. Diagn. Virol. 8(3):199-208.
Bonetta, L. (2004) “RNAi: Silencing never sounded better” Nature Methods 1(1):79-86.
Calegari, F. et al. (2002) “Tissue-specific RNA interference in postimplantation mouse embryos with endoribonuclease-prepared short interfering RNA”, Proc. Natl. Acad. Sci. USA 99(22):14236-40.
Chae, S-S. et al. (2004) “Requirement for sphingosine 1-phosphate receptor-1 in tumor angiogenesis demonstrated by in vivo RNA interference”, J. Clin. Invest 114:1082-1089.
Chiu, Y.-L. et al. (2002) “RNAi in Human Cells: Basic Structural and Functional Features of Small Interfering RNA”, Mol. Cell. 10:549-561.
Clackson, T. et al. (1991) “Making Antibody Fragments Using Phage Display Libraries” Nature 352:624-628.
Clawson, G. A. et al. (2004) “Inhibition of papilloma progression by antisense oligonucleotides targeted to HPV11 E6/L7 RNA”, Gene Ther. 11(17):1331-1341.
deBoer, H. A. et al. (1983) “The tac promoter: a functional hybrid derived from the trp and lac promoters” Proc. Natl. Acad. Sci. U.S.A. 80(1):21-25.
de Fougerolles, A. et al. (2005) “RNA Interference In Vivo: Toward Synthetic Small Inhibitory RNA-Based Therapeutics”, Methods Enzymol. 392:278-296.
Elbashir, S. M. et al. (2001) “Duplexes of 21-nucleotide RNAs mediate RNA interference in cultured mammalian cells”, Nature 411:494-498,
Eihauer, A. et al. (2001) “The FLAG™ Peptide, a Versatile Fusion Tag for the Purification of Recombinant Proteins” J. Biochem Biophys Methods 49:455-65.
Fattal et al. (1998) “Biodegradable Polyalkylcyanoacrylate Nanoparticles for the Delivery of Oligonucleotides”, J. Control Release 53(1-3): 137-43.
Fuchs, U. et al. (2004) “Silencing of Disease-related Genes by Small Interfering RNAs”, Curr. Mol. Med. 4:507-517.
Gaultier, C. et al. (1987) “α-DNA IV: α-anomeric and β-anomeric tetrathymidylates covalently linked to intercalating oxazolopyridocarbazole. Synthesis, physicochemical properties and poly (rA) binding”, Nucleic Acids. Res. 15:6625-6641.
Godard, G. et al. (1995) “Antisense effects of cholesterol-oligodeoxynucleotide conjugates associated with poly(alkylcyanoacrylate) nanoparticles”, Eur. J. Biochem. 232(2):404-10.
Goodchild, J. (2004) “Oligonucleotide therapeutics: 25 years agrowing”, Curr. Opin. Mol. Ther. 6(2):120-128.
Gish, W. et al. (1993) “Identification of protein coding regions by database similarity search” Nature Genetics 3:266-272.
Gupta, S. et al. (2004) “From the Cover: Inducible, reversible, and stable RNA interference in mammalian cells”, PNAS 101:1927-1932.
Higgins, D. G. et al. (1996) “Using CLUSTAL for multiple sequence alignments” Methods Enzymol. 266:383-402.
Holliger, P. et al. (1993) “‘Diabodies’: small bivalent and bispecific antibody fragments” Proc. Natl. Acad. Sci. USA 90:6444-6448.
Hutvagner, G. and Zamore, P. D. (2002) “RNAi: nature abhors a double-strand”, Curr. Opin. Genet. Dev. 12:225-232.
Ichim, T. E. et al. (2004) “RNA Interference: A Potent Tool for Gene-Specific Therapeutics”, Am. J. Transplant 4:1227-1236.
Inoue, H. et al. (1987) “Synthesis and hybridization studies on two complementary nona(2′-O-methyl)ribonucleotides”, Nucleic Acids Res. 15:6131-6148.
Inoue, H. et al. (1987a) “Sequence-dependent hydrolysis of RNA using modified oligonucleotide splints and RNase H”, FEBS Lett. 215:327-330.
Jacque J.-M. et al. (2002) “Modulation of HIV-1 replication by RNA interference”, Nature 418:435-438.
Jana, S. et al. (2004) “RNA Interference: Potential Therapeutic Targets”, Appl. Microbiol. Biotechnol. 65:649-657.
Jiamg, M. et al. (2004) “Gel-Based Application of siRNA to Human Epithelial Cancer Cells Induces RNAi-Dependent Apoptosis”, Oligonucleotides 14(4):239-48.
Jones, C. et al. (1995) “Current Trends in Molecular Recognition and Bioseparation” J. of Chromatography A. 707:3-22.
Keller, G. H., M. M. Manak (1987) DNA Probes, Stockton Press, New York, N.Y., pp. 169-170.
Kim, B. et al. (2004) “Inhibition of Ocular Angiogenesis by siRNA Targeting Vascular Endothelial Growth Factor Pathway Genes: Therapeutic Strategy for Herpetic Stromal Keratitis”, American Journal of Pathology 65:2177-2185.
Kohler, G. et al. (1975) “Continuous Cultures of Fused Cells Secreting Antibody of Predefined Specificity” Nature 256(5517):495-497.
Kusterbeck, A. W. et al. (1990a) “A Continuous Flow Immunoassay for Rapid and Sensitive Detection of Small Molecules” Journal of Immunological Methods 135(1-2): 191-197.
Kusterbeck, A. W. et al. (1990) “Antibody-Based Biosensor for Continuous Monitoring” In Biosensor Technology, R. P. Buck et al., eds., Marcel Dekker, N.Y. pp. 345-350.
Lambert, G. et al. (2001) “Nanoparticulate systems for the delivery of antisense oligonucleotides”, Drug Deliv. Rev. 47(1): 99-112.
Lee, N. S. et al. (2002) “Efficient delivery of siRNA for inhibition of gene expression in postnatal mice”, Nature Biotechnol. 20:500-505.
Lewis, D. L. (2002) “Efficient delivery of siRNA for inhibition of gene expression in postnatal mice”, Nature Genetics 32:107-108.
Ligler, F. S. et al. (1992) “Drug Detection Using the Flow Immunosensor” In Biosensor Design and Application, J. Findley et al., eds., American Chemical Society Press, pp. 73-80.
McCaffrey, A. P. et al. (2002) “RNA Interference in Adult Mice”, Nature 418(6893):38-39.
McManus, M. T. et al. (2002) “Gene silencing using micro-RNA designed hairpins”. RNA 8:842-850.
Maniatis, J.-M. et al. (1982) Molecular Cloning. A Laboratory Manual, Cold Spring Harbor Laboratory, New York.
Margolin, W. (2000) “Green Fluorescent Protein as a Reporter for Macromolecular Localization in Bacterial Cells” Methods 20:62-72.
Marks, J. D. et al. (1991) “By-Passing Immunization: Human Antibodies from V-Gene Libraries Displayed on Phage” J. Mol. Biol. 222(3):581-597.
Melton, D. A. et al. (1984) “Efficient In Vitro Synthesis of Biologically Active RNA and RNA Hybridization Probes From Plasmids Containing a Bacteriophage SP6 Promoter” Nuc. Acids Res. 12:7035-7036.
Miyagishi, M. and Taira, K. (2002) “U6 Promotor-Driven siRNAs with Four Uridine 3′ Overhangs Effectively Suppress Targeted Gene Expression in Mammalian Cells”, Nature Biotechnol. 20:497-500.
Morrison, S. L. et al. (1984) “Chimeric Human Antibody Molecules: Mouse Antigen-Binding Domains with Human Constant Region Domains” Proc. Natl. Acad. Sci. USA 81:6851-6855.
Ogert, R. A. et al. (1992) “Detection of Cocaine Using the Flow Immunosensor” Analytical Letters 25:1999-2019.
Paddison, P. J. et al. (2002) “Short hairpin RNAs (shRNAs) induce sequence-specific silencing in mammalian cells”, Genes Dev. 16:948-958.
Pardridge, W. M. (2004) “Intravenous, non-viral RNAi gene therapy of brain cancer”, Expert Opin. Biol. Ther. 4(7):1103-1113.
Paul, C. P. et al. (2002) “Effective expression of small interfering RNA in human cells”, Nature Biotechnol. 20:505-508.
Pearson, W. R. et al. (1988) “Improved Tools for Biological Sequence Comparison” Proc. Natl. Acad. Sci. USA 85(8):2444-2448.
Pietu, G. et al. (1996) “Novel gene transcripts preferentially expressed in human muscles revealed by quantitative hybridization of a high density cDNA array” Genome Research 6(6):492-503.
Pluckthun, A. (1994) In The Pharmacology of Monoclonal Antibodies, Vol. 113:269-315, Rosenburg and Moore eds. Springer-Verlag, New York.
Puig, O. et al. (2001) “The Tandem Affinity Purification (TAP) Method: A General Procedure of Protein Complex Purification” Methods 24:218-29.
Ryther, R. C. C. et al. (2005) “siRNA therapeutics: big potential from small RNAs”, Gene Ther. 12:5-11.
Sambrook, J. et al. (1989) Molecular Cloning, A Laboratory Manual, Second Edition, Cold Spring Harbor Press, N.Y., pp. 9.47-9.57.
Sassenfeld, H. M. (1990) “Engineering Proteins for Purification” TibTech 8:88-93.
Schena, M. et al. (1995) “Quantitative Monitoring of Gene Expression Patterns With a Complementary DNA Microarray” Science 270:467-470.
Schena, M. et al. (1996) “Parallel human genome analysis: microarray-based expression monitoring of 1000 genes” Proc. Natl. Acad Sci. U.S.A. 93(20):10614-10619.
Schena, M. (1996) “Genome analysis with gene expression microarrays” BioEssays 18(5):427-431.
Schwab et al. (1994) Ann. Oncol. 5 Suppl. 4:55-58.
Sharp, P. A. (2001) “RNA interference”, Genes Dev. 15:485-490.
Sheibani, N. (1999) “Prokaryotic Gene Fusion Expression Systems and Their Use in Structural and Functional Studies of Proteins” Prep. Biochem. & Biotechnol. 29(1):77-90.
Shen, W.-G. (2004) “RNA Interference and its Current Application in Mammals”, Chin. Med. J. (Engl) 117:1084-1091.
Skerra, A. et al. (1999) “Applications of a Peptide Ligand for Streptavidin: the Strep-tag” Biomolecular Engineering 16:79-86.
Smith, C. (1998) “Cookbook for Eukaryotic Protein Expression: Yeast, Insect, and Plant Expression Systems” The Scientist 12(22):20.
Smyth, G. K. et al. (2000) “Eukaryotic Expression and Purification of Recombinant Extracellular Matrix Proteins Carrying the Strep II Tag” Methods in Molecular Biology 139:49-57.
Soutschek, J. et al. (2004) “Therapeutic silencing of an endogenous gene by systemic administration of modified siRNAs”, Nature 432:173-178.
Suggs, S. V. et al. (1981) ICN-UCLA Symp. Dev. Biol. Using Purified Genes, D. D. Brown [ed.], Academic Press, New York, 23:683-693.
Sui, G. et al. (2002) “A DNA vector-based RNAi technology to suppress gene expression in mammalian cells”, Proc. Natl. Acad. Sci. USA 99(6):5515-5520.
Takaku, H. (2004) “Gene silencing of HIV-1 by RNA interference”, Antivir Chem. Chemother 15:57-65.
Thompson, J. et al. (1994) “Clustal-W: improving the sensitivity of progressive multiple sequence alignment through sequence weighting, position specific gap penalties and weight matrix choice” Nucleic Acids Res. 22(2):4673-4680.
Tuschl, T. (2002) “Expanding small RNA interference”, Nature Biotechnol. 20:446-448.
Unger, T. F. (1997) “Show Me the Money: Prokaryotic Expression Vectors and Purification Systems” The Scientist 11(17):20.
Villa-Kamaroff, L. et al. (1978) “A bacterial clone synthesizing proinsulin” Proc. Natl. Acad. Sci. U.S.A. 75(8):3727-3731.
Wadhwa, R. et al. (2004) “Know-how of RNA interference and its applications in research and therapy”, Mutat. Res. 567:71-84.
Wong, T. K., Neumann, E. (1982) Electric field mediated gene transfer” Biochim. Biophys. Res. Commun., 107(2):584-587.
Xia et al. (2002) “siRNA-Mediated Gene Silencing in vitro and in vivo”, Nature Biotechnol. 20(10):1006-10.
Yamamoto, T. et al. (1980) “Identification of a functional promoter in the long terminal repeat of Rous sarcoma virus” Cell 22(3):787-797.
Yu, J.-Y. et al. (2002) “RNA interference by expression of short-interfering RNAs and hairpin RNAs in mammalian cells”, Proc. Natl. Acad. Sci. USA 99(9):6047-6052.
Yuan, B. et al. (2004) “siRNA Selection Server: an automated siRNA oligonucleotide prediction server”, Nucleic Acids Research, Vol. 32, W130-W134, Web Server issue.
Zapata, G. et al. (1995) “Engineering linear F(ab′)₂ fragments for efficient production in Escherichia coli and enhanced antiproliferative activity” Protein Eng. 8(10):1057-1062.
Zheng, B. J. (2004) “Prophylactic and therapeutic effects of small interfering RNA targeting SARS-coronavirus”, Antivir. Ther. 9:365-374.

1. A composition of matter comprising:
a) an isolated, purified, and/or recombinant polypeptide comprising SEQ ID NO: 2 or an isolated, purified and/or recombinant polypeptide that is at least 93.15% identical to the polypeptide of SEQ ID NO: 2 over the full length of SEQ ID NO: 2;
b) a fragment of the polypeptide set forth in SEQ ID NO: 2 or a fragment of SEQ ID NO: 2 that is “from Y to Z”, wherein Y is the N-terminal amino acid of the specified sequence and Z, is the C-terminal amino acid of the specified sequence with the proviso that at least one of the amino acids found at positions 366 through 392 is contained within said fragment;
c) a polypeptide according to any one of embodiments a) or b) that further comprises a heterologous polypeptide sequence;
d) a composition comprising a carrier and a polypeptide as set forth in any one of a), b) or c), wherein said carrier is an adjuvant or a pharmaceutically acceptable excipient;
e) a polynucleotide sequence: i) encoding a polypeptide comprising SEQ ID NO: 2; ii) encoding one or more polypeptide fragment of SEQ ID NO: 2 as set forth in (b); or iii) encoding a polypeptide as set forth in (b) or (c);
f) a polynucleotide sequence that is at least 91.50% identical to SEQ ID NO: 1 (over the full length of SEQ ID NO: 1);
g) a polynucleotide sequence comprising SEQ ID NO: 1, 3 or 4;
h) a polynucleotide sequence that is at least 8 consecutive nucleotides of a polynucleotide sequence as set forth in (e), (f) or (g) or a span of nucleotides as set forth in in Table 3 or 4;
i) a polynucleotide that is fully complementary to the polynucleotides set forth in (e), (f), (g) or (h);
j) a polynucleotide that hybridizes under low, intermediate or high stringency with a polynucleotide sequence as set forth in (e), (f), (g), (h) or (i);
k) a genetic construct comprising a polynucleotide sequence as set forth in (e), (f), (g), (h), (i), or (j);
l) a vector comprising a polynucleotide or genetic construct as set forth in (e), (f), (g), (h), (i), (j), (k) or (l);
m) a host cell comprising a vector as set forth in (l), a genetic construct as set forth in (k), or a polynucleotide as set forth in any one of (e), (f), (g), (h), (i) or (j);
n) a probe or primer comprising a polynucleotide according to (g), (h), (i), (j), (k) or (l) and, optionally, a label or marker;
o) an antisense nucleic acid comprising a sequence fully complementary to the polynucleotide of SEQ ID NO: 1, a fragment of SEQ ID NO: 1 that includes or spans a least one nucleotide at positions 1095 to 1197 of SEQ ID NO: 1 and is at least 8 nucleotides in length, or a span of nucleotides as set forth in Table 3 or Table 4; or
p) a siRNA molecule comprising SEQ ID NO: 3 or 4.

2. A method of creating, reordering or revising a classification system of liver transplant patients comprising: (a) analyzing a hepatic biological sample of a potential liver transplant patient for the presence or absence of a polynucleotide comprising SEQ ID NO: 1 or a polypeptide comprising SEQ ID NO: 2; (b) categorizing the potential liver transplant patient on the basis of the presence or absence or said polynucleotide or polypeptide in said hepatic biological sample; and (c) assigning a potential liver transplant patient a high priority on a liver transplantation list or a classification system of liver transplant patients if said polynucleotide or said polypeptide is present in the hepatic biological sample of said potential liver transplant patient of reordering or revising the position of said potential liver transplant patient in the classification system or on a transplantation list such that the patient is more likely to receive a liver transplant or that the priority of the patient on a liver transplantation list or in a classification system of liver transplant patients is increased if said polynucleotide or said polypeptide is present in the biological sample of said patient.

3. A method of reducing the expression of the polypeptide of SEQ ID NO: 2 or the polynucleotide of SEQ ID NO: 1 in a cell or in the liver of an individual comprising the administration of an inhibitory polynucleotide, that reduces the expression of the polypeptide of SEQ ID NO: 2 or the polynucleotide of SEQ ID NO: 1 within the cell or individual, to a cell or individual.

4. The method according to claim 3, wherein said inhibitory polynucleotide is an antisense polynucleotide, a small interfering RNA (siRNA) a micro-RNA (miRNA), functional small-hairpin RNA (shRNA), or other dsRNA.

5. A method of identifying an individual at risk for terminal liver failure comprising the detection of: a) a polynucleotide comprising SEQ ID NO: 1; b) a polypeptide comprising SEQ ID NO: 2; or 3) an antibody that specifically binds to the polypeptide of SEQ ID NO: 2 in a biological sample obtained from said individual, wherein the presence of said polynucleotide, the presence of said antibody or the presence of said polypeptide is associated with liver failure (or end stage liver failure).

6. The method according to claim 5, wherein said method comprises the detection of the polypeptide of SEQ ID NO: 2 and comprises the detection of said polypeptide with an antibody that specifically binds to the polypeptide of SEQ ID NO: 2 and does not immunoreact with known alpha-1-antitrypsin polypeptides.

7. The method according to claim 5, wherein said method comprises the detection of the polynucleotide of SEQ ID NO: 1 and comprises the detection of said polynucleotide with a probe or primer that hybridizes with a target segment of SEQ ID NO: 1 that includes or spans a least one nucleotide at positions 1095 and 1197 of SEQ ID NO 1.
